SEQUENCE LISTING (21 sequences)

SEQ ID NO: 1 - protein, 243 aa, Acipenser baerii
MTMSAFSFLLALAIAGTHVSHSEESPTSEENTMKNIESKVNDLWQSLLHPVAFVAKDAELVYASCEMKPSTKLEEGKPQVTGKVLFKQAYPQGRLESIINLEGFPKTSNQSRAIHIHEFGDLSDGCDAAGGHFNPFKVNHPRHPGDFGNFLPKNSQIKTLKKNIQATMFGPNSFLSRSVVIHELKDDLGKGDNPASLLNGNAGKRLACCVIGISNKNLWEKTSQSLTSSKKKRNARGLANKQA

SEQ ID NO: 2 - protein, 155 aa, Acipenser baerii
MVLKAVCVLKGTGDVCGTVHFVQEKEAGPVKLTGQITGLTPGEHGFHVHAFGDNTNGCASAGPHFNPLGKTHGAPQDEIRHIGDLGNVIAGDDKVAIINIEDKLITLSGAYSIIGRTMVIHEKADDLGKGGNDESLVTGNAGGRLACGVIGIAQS

SEQ ID NO: 3 - protein, 207 aa, Acipenser baerii
MAPLAPLASCILLLLWLAAPSRACTCAPLHPQTAFCSSDFIIRAKFVGTAEVNQTALSQRYEIKMTKMFKGFSALGDASDIRFVYTPTAESVCGYFHRSQNRSEEFLIAGKLRNGHLHINTCSYVVPWNSLSSSQRRGFTKTYAAGCEECTVFSCSSIPCKLQNDTHCLWTDQFLTGTDKGFQSRHLACLPREPGICTWQSLRTRMA

SEQ ID NO: 4 - protein, 155 aa, Acipenser baerii
MAEGKVTVLTILPEKFNLHLENYKKPNLLYCYNGGYFLRVLPNGAVDGIRDRSDKHIQLQVTAENVGVVSIKGLEAGRYLAMSTDGQLCGSQTLTDECFFLETLEENHYATYKAQKYQDRNWYVGIKKNGRCKSGEKTHIGQKAILFLPLSASSD

SEQ ID NO: 5 - protein, 155 aa, Acipenser baerii
MAAGGITTFPTVPDDGGSSTFPPGNFKEPKRLYCKNGGYFLRINPDGRVDGIREKDDPRIKLQLQAESIGVVSIKGVSANRYLAMNEDGRLFGSKCTTDECFFFERLESNNYNTYRSQKYPDWYVALKRTGQFKSGSKTGPGQKAILFLPMSAKS

SEQ ID NO: 6 - protein, 219 aa, Acipenser baerii
MEDHLSYNKHQAYCHNCIESGNSSNSIKVRKMSTSRQLLVFTIALTVYIVDVANSIASAETLCGGELVDTLQFVCGDRGFYFSKPPSRSNIRRSQKGIVEVCCYSSCDLRLLEMYCAKPAKSERDVSSTQFQVIPLALNKDVSKKPIIAKYSKYELWQKKAAQRLRRGVPSILKARKFRRHAEEIKALEQTRFHRPLITLPSKQPVTVKPLTENYASKK

SEQ ID NO: 7 - protein, 2285 aa, Acipenser baerii
MKIYLSLIVLMICASSVECNQRGLFPAILNLASNAEITANASCGETGPEMFCKLVEHVPGRPIRNPQCRVCDSTSPNPRERHPISNAIDGTNNWWQSPSIKNGRQNHWVTITLDLRQVFQVAYIIIKAANSPRPGNWILERSIDGIEFQPWQYHAISDTECLTRYNVTPRIGPPTYKRDDEVICTSYYSRLVPLEHGEIHTSLINGRPSADDLTPALLEFTSARYIRLRLQRIRTLNADLMTLSYRDPKDVDPIVTRRKLQCVCEHNTCGESCNKCCPGYHQKPWKPGTLSVGNTCEKCNCHNKTEDCYYNQTVASSMMSMNIDGQFVGGGVCIDCTQNTAGINCETCVDGNYRPHKVSPYDENPCVECGCDLNGSQHPVCIKDDNHADLQNGLLPGKCHCKEGYAGEKCDRCSFGYSGYPYCVKCNCSLIGSIHFDPCVEQCTCKENVMGDNCDLCKRGYYNLQKSSPEGCTECFCFGVSDVCESISWPLTQVLNIDDWLMPVRPIPITSGTRFDHDDSNHISKESRILPPQSSWSAPESFLGNKLTAYGGYLNYTLSYDVPVESLHLQLMSNFDVVIEGNGKTLHTRFNAQLLLQPYKEQTVAVEMLPRNFMDFYTSRAIERDRLMTVLANLTPTVNAIDLKPAMDVEHCECPWGYTGTSCESCSPGHYRVDGILFGGICRLCECNGHATECDIHGVCSDCKHNTIGPHCEQCMPGFYGDPSDGMPDDCQVCACPLNIPSNNFSSTCHLDSAGEVMCDQCVPGYEGPRCERCANGYYGDPTVPGELCVPCDCNGNVNPLEPGHCDTRTGECLKCVENTAGSHCERCADGYFGDAIVAKNCQDCGCHVNSSYSSVCDLESGKCKCRPNVTGEKCDQCMPGHFGLSSGLGCQTCNCNLAGSLSDTCDDEGQCPCVPGVAGEKCDRCARGFYDFQDDGCTPCDCAHTHNNCNTETGQCICPPHTAGVKCELCEANHWGHDSKLGCKPCDCSLVGSSSSQCDLSSGWCQCNSEFGAEKCNECALGFRAYPECTACNCSIIGTREEYCDEKQGVCGCDKDSSTCSCKVNVVGPGCDECKLGTFALSADDPLGCSPCFCFGVSETCEELGGLVRIPIILTPDQETLHVVSQSDLSGTLDGVFLQSPDILLDAALVKQSLHTEPFYWRLPEQFQGNKLLAYGGKLRYTAAFYALEGSGISNFEPQLLIKGGRTSKLNIYRDVPAPDNGVETSQEIDLKEREWKYFNSVSDQAVSHSDFMSVLSNIQYILIKASYGSDLQQSRISNISLEIAVDSDEMNAGRETARGIERCKCPSGYAGLSCQECAPGFYRQSLTELNVRGPRPLIEPSCNDNCTGVLLNDLENLELSMQSVNLTGVILAPYSLLASLENTTDEIKMLLSPERNSSFLLKKAEEQLSGLTKDIDQLHQKTTQAYGDGQDLNKSTERMVNQSQELLDSITKVQTAIQALVELGSNLNNTLGNNLVLTNSTQLLDEVSAVLEKMRNKAFTQQHQNATHEFKAAEALLLQVQREFQKPQKDVMELKERIVTILSEHSSKLQDAQNLVNESFTKANETNHILHAMSSNLAVFYDKKQNVSSNHFLTTEMIEEAQATMTEGLNIATDVVNATSQLEGHKDELALWNPKLRKQVDNLVMQMTKRSVLDLVYKAEDHAAGLKKLADSLHNVLSGVRNVSFNATNAAQANSNIKINIEKAERLAEQANSTVAAAWSLALFADHSLKDAGAKSLQQSSRLLSEAADLNNKTNGLISDLNGLKEQVDKIRYNAQNISKQLSEPLQLLSSIPNNVSVKVLEAKEHAMGANVSAVAALQHLEDFSQKLEESSSAMIRANETVKRTNELFSDSAKTANAAEKKVQEVETQTNLLFERLKPLKMLEDTLSRNLSEIKELIDQARKQAASIKVAVAAEQDCVRAYKPDISSSNYNTLTLIVKTSESDNLLFYLGSSTNVDFMALEMRRGKVSFLWDVGSGFAKLEYPDIQINNDKWHRIHATRFGKTGSLTVQELKSSQTPTVKTATSPGTSTVLDVNKSTLVFVGGLGGQIKKSSAVKMTHFKGCMGGASLNGNNIGLWNYAEREGKCRGCFMSPQDEDTSFHFDGSGYSVVEKALRSTVTQIVMLFKTFSPNGLLLYLASNGTRDFTSIELVDGKVRLTFELGSGPLSLITDKTYNSGNWYKIAFHRTKQKGYLAVMDAYIPTNRETKEGTTSGSASDLNRSDKDPIYIGGLPRSRPVRRQLVTRSYVGCIKNLEIARSNFDLLKESYGVKKGCVLEHGTEPWY

SEQ ID NO: 8 - protein, 315 aa, Acipenser sp.
MYLFPQIWITAFIYPCLLFCFTFVEHSCGLGNVVTDDVNKIPILKGNIPNDYIIKVRYVPQSEELNDICWLMLNIYELQLSLTSLAVKFAETSSNKENITVLIDMVMKMRRPFQYEEEVIDDYHCHYQDGNFNTSVYFDYLKKMIETYELHERQFISPDCVSPPCPTSETTTLVAGSITIATELLIMNTTACVSASDCNTQETRRDKNTEAKTNTQGAEKLHIPLYLLLIPVFGILLVLTWKGHITSCCRTIVLKGVDVASNTWVTLYMLLPDEAVLYKSTYSSFIWSEGQKLDSCSVELLSETRVCKTSTPEIQ

SEQ ID NO: 9 - DNA, 729 bp, Synthetic construct
  source 1..729: mol_type = other DNA
atgactatgtctgctttttcttttttgttggctttggctattgctggtactcatgtttctcattctgaagaatctccaacttctgaagaaaatactatgaaaaatattgaatctaaagttaatgatttgtggcaatctttgttgcatccagttgcttttgttgctaaagatgctgaattggtttatgcttcttgtgaaatgaaaccatctactaaattggaagaaggtaaaccacaagttactggtaaagttttgtttaaacaagcttatccacaaggtagattggaatctattattaatttggaaggttttccaaaaacttctaatcaatctagagctattcatattcatgaatttggtgatttgtctgatggttgtgatgctgctggtggtcattttaatccatttaaagttaatcatccaagacatccaggtgattttggtaattttttgccaaaaaattctcaaattaaaactttgaaaaaaaatattcaagctactatgtttggtccaaattcttttttgtctagatctgttgttattcatgaattgaaagatgatttgggtaaaggtgataatccagcttctttgttgaatggtaatgctggtaaaagattggcttgttgtgttattggtatttctaataaaaatttgtgggaaaaaacttctcaatctttgacttcttctaaaaaaaaaagaaatgctagaggtttggctaataaacaagct

SEQ ID NO: 10 - DNA, 465 bp, Synthetic construct
  source 1..465: mol_type = other DNA
atggttttgaaagctgtttgtgttttgaaaggtactggtgatgtttgtggtactgttcattttgttcaagaaaaagaagctggtccagttaaattgactggtcaaattactggtttgactccaggtgaacatggttttcatgttcatgcttttggtgataatactaatggttgtgcttctgctggtccacattttaatccattgggtaaaactcatggtgctccacaagatgaaattagacatattggtgatttgggtaatgttattgctggtgatgataaagttgctattattaatattgaagataaattgattactttgtctggtgcttattctattattggtagaactatggttattcatgaaaaagctgatgatttgggtaaaggtggtaatgatgaatctttggttactggtaatgctggtggtagattggcttgtggtgttattggtattgctcaatct

SEQ ID NO: 11 - DNA, 621 bp, Synthetic construct
  source 1..621: mol_type = other DNA
atggctccattggctccattggcttcttgtattttgttgttgttgtggttggctgctccatctagagcttgtacttgtgctccattgcatccacaaactgctttttgttcttctgattttattattagagctaaatttgttggtactgctgaagttaatcaaactgctttgtctcaaagatatgaaattaaaatgactaaaatgtttaaaggtttttctgctttgggtgatgcttctgatattagatttgtttatactccaactgctgaatctgtttgtggttattttcatagatctcaaaatagatctgaagaatttttgattgctggtaaattgagaaatggtcatttgcatattaatacttgttcttatgttgttccatggaattctttgtcttcttctcaaagaagaggttttactaaaacttatgctgctggttgtgaagaatgtactgttttttcttgttcttctattccatgtaaattgcaaaatgatactcattgtttgtggactgatcaatttttgactggtactgataaaggttttcaatctagacatttggcttgtttgccaagagaaccaggtatttgtacttggcaatctttgagaactagaatggct

SEQ ID NO: 12 - DNA, 945 bp, Synthetic construct
  source 1..945: mol_type = other DNA
atgtatttgtttccacaaatttggattactgcttttatttatccatgtttgttgttttgttttacttttgttgaacattcttgtggtttgggtaatgttgttactgatgatgttaataaaattccaattttgaaaggtaatattccaaatgattatattattaaagttagatatgttccacaatctgaagaattgaatgatatttgttggttgatgttgaatatttatgaattgcaattgtctttgacttctttggctgttaaatttgctgaaacttcttctaataaagaaaatattactgttttgattgatatggttatgaaaatgagaagaccatttcaatatgaagaagaagttattgatgattatcattgtcattatcaagatggtaattttaatacttctgtttattttgattatttgaaaaaaatgattgaaacttatgaattgcatgaaagacaatttatttctccagattgtgtttctccaccatgtccaacttctgaaactactactttggttgctggttctattactattgctactgaattgttgattatgaatactactgcttgtgtttctgcttctgattgtaatactcaagaaactagaagagataaaaatactgaagctaaaactaatactcaaggtgctgaaaaattgcatattccattgtatttgttgttgattccagtttttggtattttgttggttttgacttggaaaggtcatattacttcttgttgtagaactattgttttgaaaggtgttgatgttgcttctaatacttgggttactttgtatatgttgttgccagatgaagctgttttgtataaatctacttattcttcttttatttggtctgaaggtcaaaaattggattcttgttctgttgaattgttgtctgaaactagagtttgtaaaacttctactccagaaattcaa

SEQ ID NO: 13 - DNA, 465 bp, Synthetic construct
  source 1..465: mol_type = other DNA
atggctgaaggtaaagttactgttttgactattttgccagaaaaatttaatttgcatttggaaaattataaaaaaccaaatttgttgtattgttataatggtggttattttttgagagttttgccaaatggtgctgttgatggtattagagatagatctgataaacatattcaattgcaagttactgctgaaaatgttggtgttgtttctattaaaggtttggaagctggtagatatttggctatgtctactgatggtcaattgtgtggttctcaaactttgactgatgaatgtttttttttggaaactttggaagaaaatcattatgctacttataaagctcaaaaatatcaagatagaaattggtatgttggtattaaaaaaaatggtagatgtaaatctggtgaaaaaactcatattggtcaaaaagctattttgtttttgccattgtctgcttcttctgat

SEQ ID NO: 14 - DNA, 465 bp, Synthetic construct
  source 1..465: mol_type = other DNA
atggctgctggtggtattactacttttccaactgttccagatgatggtggttcttctacttttccaccaggtaattttaaagaaccaaaaagattgtattgtaaaaatggtggttattttttgagaattaatccagatggtagagttgatggtattagagaaaaagatgatccaagaattaaattgcaattgcaagctgaatctattggtgttgtttctattaaaggtgtttctgctaatagatatttggctatgaatgaagatggtagattgtttggttctaaatgtactactgatgaatgttttttttttgaaagattggaatctaataattataatacttatagatctcaaaaatatccagattggtatgttgctttgaaaagaactggtcaatttaaatctggttctaaaactggtccaggtcaaaaagctattttgtttttgccaatgtctgctaaatct

SEQ ID NO: 15 - DNA, 657 bp, Synthetic construct
  source 1..657: mol_type = other DNA
atggaagatcatttgtcttataataaacatcaagcttattgtcataattgtattgaatctggtaattcttctaattctattaaagttagaaaaatgtctacttctagacaattgttggtttttactattgctttgactgtttatattgttgatgttgctaattctattgcttctgctgaaactttgtgtggtggtgaattggttgatactttgcaatttgtttgtggtgatagaggtttttatttttctaaaccaccatctagatctaatattagaagatctcaaaaaggtattgttgaagtttgttgttattcttcttgtgatttgagattgttggaaatgtattgtgctaaaccagctaaatctgaaagagatgtttcttctactcaatttcaagttattccattggctttgaataaagatgtttctaaaaaaccaattattgctaaatattctaaatatgaattgtggcaaaaaaaagctgctcaaagattgagaagaggtgttccatctattttgaaagctagaaaatttagaagacatgctgaagaaattaaagctttggaacaaactagatttcatagaccattgattactttgccatctaaacaaccagttactgttaaaccattgactgaaaattatgcttctaaaaaa

SEQ ID NO: 16 - DNA, 6855 bp, Synthetic construct
  source 1..6855: mol_type = other DNA
atgaaaatttatttgtctttgattgttttgatgatttgtgcttcttctgttgaatgtaatcaaagaggtttgtttccagctattttgaatttggcttctaatgctgaaattactgctaatgcttcttgtggtgaaactggtccagaaatgttttgtaaattggttgaacatgttccaggtagaccaattagaaatccacaatgtagagtttgtgattctacttctccaaatccaagagaaagacatccaatttctaatgctattgatggtactaataattggtggcaatctccatctattaaaaatggtagacaaaatcattgggttactattactttggatttgagacaagtttttcaagttgcttatattattattaaagctgctaattctccaagaccaggtaattggattttggaaagatctattgatggtattgaatttcaaccatggcaatatcatgctatttctgatactgaatgtttgactagatataatgttactccaagaattggtccaccaacttataaaagagatgatgaagttatttgtacttcttattattctagattggttccattggaacatggtgaaattcatacttctttgattaatggtagaccatctgctgatgatttgactccagctttgttggaatttacttctgctagatatattagattgagattgcaaagaattagaactttgaatgctgatttgatgactttgtcttatagagatccaaaagatgttgatccaattgttactagaagaaaattgcaatgtgtttgtgaacataatacttgtggtgaatcttgtaataaatgttgtccaggttatcatcaaaaaccatggaaaccaggtactttgtctgttggtaatacttgtgaaaaatgtaattgtcataataaaactgaagattgttattataatcaaactgttgcttcttctatgatgtctatgaatattgatggtcaatttgttggtggtggtgtttgtattgattgtactcaaaatactgctggtattaattgtgaaacttgtgttgatggtaattatagaccacataaagtttctccatatgatgaaaatccatgtgttgaatgtggttgtgatttgaatggttctcaacatccagtttgtattaaagatgataatcatgctgatttgcaaaatggtttgttgccaggtaaatgtcattgtaaagaaggttatgctggtgaaaaatgtgatagatgttcttttggttattctggttatccatattgtgttaaatgtaattgttctttgattggttctattcattttgatccatgtgttgaacaatgtacttgtaaagaaaatgttatgggtgataattgtgatttgtgtaaaagaggttattataatttgcaaaaatcttctccagaaggttgtactgaatgtttttgttttggtgtttctgatgtttgtgaatctatttcttggccattgactcaagttttgaatattgatgattggttgatgccagttagaccaattccaattacttctggtactagatttgatcatgatgattctaatcatatttctaaagaatctagaattttgccaccacaatcttcttggtctgctccagaatcttttttgggtaataaattgactgcttatggtggttatttgaattatactttgtcttatgatgttccagttgaatctttgcatttgcaattgatgtctaattttgatgttgttattgaaggtaatggtaaaactttgcatactagatttaatgctcaattgttgttgcaaccatataaagaacaaactgttgctgttgaaatgttgccaagaaattttatggatttttatacttctagagctattgaaagagatagattgatgactgttttggctaatttgactccaactgttaatgctattgatttgaaaccagctatggatgttgaacattgtgaatgtccatggggttatactggtacttcttgtgaatcttgttctccaggtcattatagagttgatggtattttgtttggtggtatttgtagattgtgtgaatgtaatggtcatgctactgaatgtgatattcatggtgtttgttctgattgtaaacataatactattggtccacattgtgaacaatgtatgccaggtttttatggtgatccatctgatggtatgccagatgattgtcaagtttgtgcttgtccattgaatattccatctaataatttttcttctacttgtcatttggattctgctggtgaagttatgtgtgatcaatgtgttccaggttatgaaggtccaagatgtgaaagatgtgctaatggttattatggtgatccaactgttccaggtgaattgtgtgttccatgtgattgtaatggtaatgttaatccattggaaccaggtcattgtgatactagaactggtgaatgtttgaaatgtgttgaaaatactgctggttctcattgtgaaagatgtgctgatggttattttggtgatgctattgttgctaaaaattgtcaagattgtggttgtcatgttaattcttcttattcttctgtttgtgatttggaatctggtaaatgtaaatgtagaccaaatgttactggtgaaaaatgtgatcaatgtatgccaggtcattttggtttgtcttctggtttgggttgtcaaacttgtaattgtaatttggctggttctttgtctgatacttgtgatgatgaaggtcaatgtccatgtgttccaggtgttgctggtgaaaaatgtgatagatgtgctagaggtttttatgattttcaagatgatggttgtactccatgtgattgtgctcatactcataataattgtaatactgaaactggtcaatgtatttgtccaccacatactgctggtgttaaatgtgaattgtgtgaagctaatcattggggtcatgattctaaattgggttgtaaaccatgtgattgttctttggttggttcttcttcttctcaatgtgatttgtcttctggttggtgtcaatgtaattctgaatttggtgctgaaaaatgtaatgaatgtgctttgggttttagagcttatccagaatgtactgcttgtaattgttctattattggtactagagaagaatattgtgatgaaaaacaaggtgtttgtggttgtgataaagattcttctacttgttcttgtaaagttaatgttgttggtccaggttgtgatgaatgtaaattgggtacttttgctttgtctgctgatgatccattgggttgttctccatgtttttgttttggtgtttctgaaacttgtgaagaattgggtggtttggttagaattccaattattttgactccagatcaagaaactttgcatgttgtttctcaatctgatttgtctggtactttggatggtgtttttttgcaatctccagatattttgttggatgctgctttggttaaacaatctttgcatactgaaccattttattggagattgccagaacaatttcaaggtaataaattgttggcttatggtggtaaattgagatatactgctgctttttatgctttggaaggttctggtatttctaattttgaaccacaattgttgattaaaggtggtagaacttctaaattgaatatttatagagatgttccagctccagataatggtgttgaaacttctcaagaaattgatttgaaagaaagagaatggaaatattttaattctgtttctgatcaagctgtttctcattctgattttatgtctgttttgtctaatattcaatatattttgattaaagcttcttatggttctgatttgcaacaatctagaatttctaatatttctttggaaattgctgttgattctgatgaaatgaatgctggtagagaaactgctagaggtattgaaagatgtaaatgtccatctggttatgctggtttgtcttgtcaagaatgtgctccaggtttttatagacaatctttgactgaattgaatgttagaggtccaagaccattgattgaaccatcttgtaatgataattgtactggtgttttgttgaatgatttggaaaatttggaattgtctatgcaatctgttaatttgactggtgttattttggctccatattctttgttggcttctttggaaaatactactgatgaaattaaaatgttgttgtctccagaaagaaattcttcttttttgttgaaaaaagctgaagaacaattgtctggtttgactaaagatattgatcaattgcatcaaaaaactactcaagcttatggtgatggtcaagatttgaataaatctactgaaagaatggttaatcaatctcaagaattgttggattctattactaaagttcaaactgctattcaagctttggttgaattgggttctaatttgaataatactttgggtaataatttggttttgactaattctactcaattgttggatgaagtttctgctgttttggaaaaaatgagaaataaagcttttactcaacaacatcaaaatgctactcatgaatttaaagctgctgaagctttgttgttgcaagttcaaagagaatttcaaaaaccacaaaaagatgttatggaattgaaagaaagaattgttactattttgtctgaacattcttctaaattgcaagatgctcaaaatttggttaatgaatcttttactaaagctaatgaaactaatcatattttgcatgctatgtcttctaatttggctgttttttatgataaaaaacaaaatgtttcttctaatcattttttgactactgaaatgattgaagaagctcaagctactatgactgaaggtttgaatattgctactgatgttgttaatgctacttctcaattggaaggtcataaagatgaattggctttgtggaatccaaaattgagaaaacaagttgataatttggttatgcaaatgactaaaagatctgttttggatttggtttataaagctgaagatcatgctgctggtttgaaaaaattggctgattctttgcataatgttttgtctggtgttagaaatgtttcttttaatgctactaatgctgctcaagctaattctaatattaaaattaatattgaaaaagctgaaagattggctgaacaagctaattctactgttgctgctgcttggtctttggctttgtttgctgatcattctttgaaagatgctggtgctaaatctttgcaacaatcttctagattgttgtctgaagctgctgatttgaataataaaactaatggtttgatttctgatttgaatggtttgaaagaacaagttgataaaattagatataatgctcaaaatatttctaaacaattgtctgaaccattgcaattgttgtcttctattccaaataatgtttctgttaaagttttggaagctaaagaacatgctatgggtgctaatgtttctgctgttgctgctttgcaacatttggaagatttttctcaaaaattggaagaatcttcttctgctatgattagagctaatgaaactgttaaaagaactaatgaattgttttctgattctgctaaaactgctaatgctgctgaaaaaaaagttcaagaagttgaaactcaaactaatttgttgtttgaaagattgaaaccattgaaaatgttggaagatactttgtctagaaatttgtctgaaattaaagaattgattgatcaagctagaaaacaagctgcttctattaaagttgctgttgctgctgaacaagattgtgttagagcttataaaccagatatttcttcttctaattataatactttgactttgattgttaaaacttctgaatctgataatttgttgttttatttgggttcttctactaatgttgattttatggctttggaaatgagaagaggtaaagtttcttttttgtgggatgttggttctggttttgctaaattggaatatccagatattcaaattaataatgataaatggcatagaattcatgctactagatttggtaaaactggttctttgactgttcaagaattgaaatcttctcaaactccaactgttaaaactgctacttctccaggtacttctactgttttggatgttaataaatctactttggtttttgttggtggtttgggtggtcaaattaaaaaatcttctgctgttaaaatgactcattttaaaggttgtatgggtggtgcttctttgaatggtaataatattggtttgtggaattatgctgaaagagaaggtaaatgtagaggttgttttatgtctccacaagatgaagatacttcttttcattttgatggttctggttattctgttgttgaaaaagctttgagatctactgttactcaaattgttatgttgtttaaaactttttctccaaatggtttgttgttgtatttggcttctaatggtactagagattttacttctattgaattggttgatggtaaagttagattgacttttgaattgggttctggtccattgtctttgattactgataaaacttataattctggtaattggtataaaattgcttttcatagaactaaacaaaaaggttatttggctgttatggatgcttatattccaactaatagagaaactaaagaaggtactacttctggttctgcttctgatttgaatagatctgataaagatccaatttatattggtggtttgccaagatctagaccagttagaagacaattggttactagatcttatgttggttgtattaaaaatttggaaattgctagatctaattttgatttgttgaaagaatcttatggtgttaaaaaaggttgtgttttggaacatggtactgaaccatggtat

SEQ ID NO: 17 - protein, 15 aa, Acipenser baerii
HFNPFKVNHPRHPGD

SEQ ID NO: 18 - protein, 11 aa, Acipenser baerii
GFHVHAFGDNT

SEQ ID NO: 19 - protein, 163 aa, Synthetic construct
MVLKAVCVLKGTGDVCGTVHFVQEKEAGPVKLTGQITGLTPGEHGFHVHAFGDNTNGCASAGPHFNPLGKTHGAPQDEIRHIGDLGNVIAGDDKVAIINIEDKLITLSGAYSIIGRTMVIHEKADDLGKGGNDESLVTGNAGGRLACGVIGIAQSHHHHHHHH

SEQ ID NO: 20 - protein, 251 aa, Synthetic construct
MTMSAFSFLLALAIAGTHVSHSEESPTSEENTMKNIESKVNDLWQSLLHPVAFVAKDAELVYASCEMKPSTKLEEGKPQVTGKVLFKQAYPQGRLESIINLEGFPKTSNQSRAIHIHEFGDLSDGCDAAGGHFNPFKVNHPRHPGDFGNFLPKNSQIKTLKKNIQATMFGPNSFLSRSVVIHELKDDLGKGDNPASLLNGNAGKRLACCVIGISNKNLWEKTSQSLTSSKKKRNARGLANKQAHHHHHHHH

SEQ ID NO: 21 - protein, 323 aa, Synthetic construct
MYLFPQIWITAFIYPCLLFCFTFVEHSCGLGNVVTDDVNKIPILKGNIPNDYIIKVRYVPQSEELNDICWLMLNIYELQLSLTSLAVKFAETSSNKENITVLIDMVMKMRRPFQYEEEVIDDYHCHYQDGNFNTSVYFDYLKKMIETYELHERQFISPDCVSPPCPTSETTTLVAGSITIATELLIMNTTACVSASDCNTQETRRDKNTEAKTNTQGAEKLHIPLYLLLIPVFGILLVLTWKGHITSCCRTIVLKGVDVASNTWVTLYMLLPDEAVLYKSTYSSFIWSEGQKLDSCSVELLSETRVCKTSTPEIQHHHHHHHH

(19) United States
(12) Patent Application Publication
(10) Pub. No.: US 2026/0108449 A1
(43) Pub. Date: Apr. 23, 2026

(54) CAVIAR POLYPEPTIDES

(71) Applicant: Shah Caviar Limited, Burscough (GB)

(72) Inventor: Kenneth Benning, Burscough (GB)

(21) Appl. No.: 19/118,325

(22) PCT Filed: Oct. 6, 2023
(86) PCT No.: PCT/GB2023/052600

§ 371 Date: Apr. 3, 2025

(30) Foreign Application Priority Data
Oct. 6, 2022 (GB) 2214683.1

(51) Int. Cl. A61K 8/64; A61K 8/66; A61Q 5/00; A61Q 19/00; C07K 14/475; C07K 14/50; C07K 14/52; C07K 14/78; C07K 14/81; C12N 9/02

(52) U.S. Cl. CPC A61K 8/64; A61K 8/66; A61Q 5/00; A61Q 19/00; C07K 14/475; C07K 14/501; C07K 14/503; C07K 14/52; C07K 14/78; C07K 14/8146; C12N 9/0089; C12Y 115/01001

(57) ABSTRACT

The present invention relates to recombinant, or synthetic caviar polypeptides and methods for their production. The invention also relates to cosmetic products comprising recombinant or synthetic polypeptides of the invention.

### FIELD OF THE INVENTION

The present invention relates to recombinant or synthetic caviar polypeptides and methods for their production. The invention also relates to cosmetic products comprising recombinant or synthetic polypeptides of the invention.

### BACKGROUND

As well as being a luxury food, caviar is widely recognised for its cosmetic benefits. Desirable properties associated with caviar include enhanced cellular metabolism, stimulation of collagen production by fibroblasts, protection against oxidative stress (and associated improved maintenance of cell membranes) and hydration of the skin. The desirable properties of caviar have resulted in widespread use of caviar and caviar extracts in applications which extend far beyond its traditional use as a luxury food. Nowhere is this more pronounced than in cosmetics and nutritional applications, where caviar and caviar extracts are now being used e.g. in skin creams, and to supplement pet foods and vodka.

Herein, the term “caviar” corresponds to traditional use of the term solely in relation to salt-treated, unfertilized eggs (oocytes) or roe from ovaries of female sturgeons. Caviar is a luxury food owing to its difficulty of production and perishable nature, and is prized for qualities including flavour, shape, colour and texture. “Sturgeon” is the common name for a large number of fish species of the family Acipenseridae, including the genera Acipenser, Huso, Scaphirhynchus and Pseudoscaphirhynchus (for more information see: www.sturgeonweb.co.uk). The term “caviar” is used more exclusively to refer to two species commonly connected with caviar production, Acipenser and Huso. For example Beluga caviar derived from wild Beluga sturgeon (Huso huso) has traditionally been much prized, but other sturgeon species are also well-known for caviar production and are increasingly farmed. These include for example Siberian sturgeon (Acipenser baerii), now being farmed for the first time for caviar production in the UK by Exmoor Caviar, and other Acipenser species.

Despite considerable and growing demand for caviar and caviar extracts, caviar production remains labor intensive and slow. A key limiting factor in the production of caviar is the many years that female sturgeons take to reach reproductive maturity. Female Siberian sturgeons commonly take 8-10 years to become mature and produce eggs; females of some species, e.g. Beluga sturgeon, take far longer, e.g. around 20 years or more. To reduce this time, some hybrid crosses have been achieved, e.g. male Huso huso sturgeon have been crossed with faster maturing species such as Acipenser sterlet (also known as Acipenser ruthenus) or baerii to provide hybrid species for farming, but even hybrid crosses take several years to reach reproductive maturity.

Whilst farming of sturgeons addresses the ecological problem of relying on wild sturgeon for caviar production and enables caviar production more widely, there remains for caviar producers the problem of the need for careful timing of harvesting of the roe, preferably very shortly before spawning, coupled with need for roe sacks to be quickly processed once extracted to avoid deterioration of the roe. This is still a delicate task usually done by hand. The roe sacks are normally gently rubbed across a sieve, either a nylon mesh or stainless steel sieve, whereby the roe eggs are separated from the membrane of the roe sack and pass through the sieve to be collected. Running cold water may be employed to aid the roe release and separation. Salt will be added to the separated roe according to requirement for taste and to aid preservation. A stabilizer may be additionally added such as E285 stabilizer. The roe, which is now caviar, is packed into containers with airtight sealing, normally under vacuum, for refrigerated storage. The caviar may be subsequently re-packaged in smaller quantities.

Ultimately, despite advancements made in the caviar industry, production remains slow, and caviar remains comparatively expensive. Established caviar production methods are also hampered by the requirement to sacrifice sturgeon during caviar harvesting, which limits the potential commercial market for products which contain caviar and caviar extracts.

There exists a need in the art for products (e.g. cosmetic products) which possess highly desirable properties associated with caviar, but are faster, cheaper and more ethical to produce, and can be produced in sufficient quantities to meet the rapidly growing demand. There also exists a need in the art for a method of producing such products.

The present invention is based on the surprising realisation that many requirements of caviar as a luxury food (e.g. flavour, shape, colour and texture) are largely irrelevant to its use in other applications (e.g. in nutritional or cosmetic products). Indeed, the appearance and texture of caviar (so desired by caviar afficionados) is often destroyed during preparation (e.g. of nutritional or cosmetic products) and can even be undesirable (e.g. in face creams). Despite this, caviar and caviar extracts remain a gold standard for many applications. Consequently, the high cost of existing products which contain caviar and caviar extracts (but are not caviar itself) is largely driven by requirements imposed by the luxury food industry, even though many of such requirements are irrelevant to other applications. Moreover, current production of products which contain caviar and caviar extracts typically requires sacrifice of sturgeon, which raises ethical questions and limits their potential commercial market.

Despite the challenges and drawbacks associated with caviar, the market remains focussed on caviar, even for applications which are unrelated to consumption as a luxury food. Indeed, the market focus on caviar has been so strong that alternatives which fail to meet the requirements placed on caviar as a luxury food have not been contemplated.

### SUMMARY OF THE INVENTION

The present invention is based on the surprising discovery of key polypeptides within caviar which possess highly desirable cellular functions, particularly for cosmetic applications. Significant technical challenges were overcome in order to identify these key caviar polypeptides; and successful identification of these key caviar polypeptides now enables their ‘animal-free’ production (e.g. by recombinant or synthetic means) without the ethical, financial or time burdens associated with caviar. Moreover, the present invention enables the production of compositions which contain a far higher concentration of these key caviar polypeptides than is achievable when using caviar, and so compositions of the invention achieve superior potency to caviar-containing compositions in the prior art.

By providing key caviar polypeptides, the present invention advantageously ‘uncouples’: (A) the highly desirable properties that are intrinsic to caviar (e.g. for use in cosmetic products), from (B) requirements placed on caviar as a luxury food. As compared to caviar:
i) Compositions of the invention may be produced more quickly than caviar (no waiting for sturgeons to reach reproductive maturity, including no waiting for oocytes to reach (at least) Stage 3);
ii) Compositions of the invention may be produced more cheaply (costs associated with in vitro production are far lower than sturgeon fishing or farming);
iii) Compositions of the invention avoid ethical concerns and provide a greater commercial market (no involvement of sturgeon in their production);
iv) Compositions of the invention may be produced with a far higher concentration of key polypeptides than is achievable when those polypeptides are provided by caviar itself. Compositions of the invention therefore exhibit superior potency.

The invention provides a composition comprising a recombinant or synthetic caviar polypeptide, wherein the caviar polypeptide is selected from: a superoxide dismutase (SOD3) polypeptide, wherein the SOD3 polypeptide comprises a polypeptide sequence having: (i) at least 70% sequence identity to the amino acid sequence of SEQ ID NO: 1; or (ii) at least 50 consecutive amino acids of SEQ ID NO: 1; a superoxide dismutase (SOD1) polypeptide, wherein the SOD1 polypeptide comprises a polypeptide sequence having: (i) at least 70% sequence identity to the amino acid sequence of SEQ ID NO: 2; or (ii) at least 50 consecutive amino acids of SEQ ID NO: 2; a tissue inhibitor of metalloproteinase 1 (TIMP1) polypeptide, wherein the TIMP1 polypeptide comprises a polypeptide sequence having: (i) at least 70% sequence identity to the amino acid sequence of SEQ ID NO: 3; or (ii) at least 50 consecutive amino acids of SEQ ID NO: 3; an acidic fibroblast growth factor (aFGF) polypeptide, wherein the aFGF polypeptide comprises a polypeptide sequence having: (i) at least 70% sequence identity to the amino acid sequence of SEQ ID NO: 4; or (ii) at least 50 consecutive amino acids of SEQ ID NO: 4; a basic fibroblast growth factor (bFGF) polypeptide, wherein the bFGF polypeptide comprises a polypeptide sequence having: (i) at least 70% sequence identity to the amino acid sequence of SEQ ID NO: 5; or (ii) at least 50 consecutive amino acids of SEQ ID NO: 5; an insulin-like growth factor 2 (IGF-2) polypeptide, wherein the IGF-2 polypeptide comprises a polypeptide sequence having: (i) at least 70% sequence identity to the amino acid sequence of SEQ ID NO: 6; or (ii) at least 50 consecutive amino acids of SEQ ID NO: 6; and a laminin polypeptide, wherein the laminin polypeptide comprises a polypeptide sequence having: (i) at least 70% sequence identity to the amino acid sequence of SEQ ID NO: 7; or (ii) at least 50 consecutive amino acids of SEQ ID NO: 7.

In some embodiments, the composition comprises two or more recombinant or synthetic caviar polypeptides. In some embodiments, the composition comprises two or more recombinant or synthetic caviar polypeptides selected from: SOD3, SOD1 and TIMP1. In some embodiments, the composition comprises SOD3, SOD1 and TIMP1. In some embodiments, the composition comprises SOD3, SOD1, TIMP1 and SCF.

In some embodiments, the composition further comprises a recombinant or synthetic stem cell factor (SCF) polypeptide, wherein the SCF polypeptide comprises a polypeptide sequence having: (i) at least 70% sequence identity to the amino acid sequence of SEQ ID NO: 8; or (ii) at least 50 consecutive amino acids of SEQ ID NO: 8.

In some embodiments, the composition comprises two or more recombinant or synthetic caviar polypeptides selected from: SOD3, SOD1 and SCF. In some embodiments, the composition comprises SOD3 and SOD1. In some embodiments, the composition comprises SOD3 and SOD1. In some embodiments, the composition comprises SOD3 and SCF. In some embodiments, the composition comprises SOD1 and SCF. In some embodiments, the composition comprises SOD3, SOD1 and SCF.

In some embodiments, the composition comprises SOD3, SOD1 and SCF, and a sodium phosphate buffer.

In some embodiments, the composition further comprises collagen.

In some embodiments, the composition is a cosmetic composition. In some embodiments, the cosmetic composition is selected from: skin moisturizer, perfume, lipstick, fingernail polishes, eye and/or facial makeup preparation, shampoo, hair colouring preparations, toothpaste, and deodorant.

The invention also provides an isolated nucleic acid which encodes one or more (e.g. 1, 2, 3, 4, 5, 6 or 7) caviar polypeptide(s) as defined above.

The invention also provides an isolated nucleic acid which encodes SCF as defined above.

In some embodiments, the nucleic acid is codon optimized for expression in a eukaryotic cell. In some embodiments, the eukaryotic cell is a yeast cell. In some embodiments: the nucleic acid encoding SOD3 has at least 70% sequence identity to SEQ ID NO: 9; the nucleic acid encoding SOD1 has at least 70% sequence identity to SEQ ID NO: 10; the nucleic acid encoding TIMP1 has at least 70% sequence identity to SEQ ID NO: 11; and the nucleic acid encoding SCF has at least 70% sequence identity to SEQ ID NO: 12.

The invention also provides a vector comprising the isolated nucleic acid as described above. In some embodiments, the vector further comprises: (a) an origin of replication; (b) a promoter sequence operably linked to said nucleic acid; and/or (c) a reporter gene.

The invention also provides a recombinant cell engineered to express a recombinant or synthetic caviar polypeptide as defined above.

The invention also provides a recombinant cell transformed with the vector as described above. In some embodiments, the recombinant cell is a P. pastoris cell.

The invention also provides a method of producing one or more recombinant caviar polypeptides as described above, the method comprising: (a) culturing a recombinant cell as described above in a suitable culture medium; and (b) allowing expression of the recombinant caviar polypeptide.

The invention also provides a method of producing one or more synthetic caviar polypeptides as described above, wherein the synthetic caviar polypeptides are produced by: (a) cell-free protein synthesis; or (b) solid-phase chemical synthesis.

The invention also provides a method of producing a cosmetic product, the method comprising combining: (a) one or more recombinant or synthetic caviar polypeptides as described above, optionally in combination with recombinant or synthetic SCF as described above; with (b) ingredients for a cosmetic product.

The invention also provides use of a composition as described above for improving the appearance of skin and/or hair.

The invention also provides a method of improving the appearance of skin, the method comprising administering to the skin of a subject a composition as described above.

The invention also provides a method of improving the appearance of hair or a subject, the method comprising administering to the hair of a subject a composition as defined above.

The invention also provides a cosmetic method comprising administering a composition as described above to the skin and/or hair of a subject. The subject is typically a mammal. In some embodiments, the subject is a human. In some embodiments, the subject is an animal e.g. a domestic pet, such as a dog, cat or horse.

Compositions of the invention are preferably animal-free. In some embodiments, compositions of the invention are vegan-friendly.

A “cosmetic product” refers to a product intended to be rubbed, poured, sprinkled, or sprayed on, introduced into, or otherwise applied to the human body for cleansing, beautifying, promoting attractiveness, or altering the appearance. Included in this definition are products such as skin moisturizers, perfumes, lipsticks, fingernail polishes, eye and facial makeup preparations, shampoos, hair colouring preparations, toothpastes, and deodorants, as well as any material intended for use as a component of a cosmetic product. In one embodiment, the cosmetic product is a finished cosmetic product.

Cosmetic products of the invention comprise one or more recombinant or synthetic caviar polypeptide(s) of the invention, and typically comprise one or more ingredients for a cosmetic product. Typical ingredients for a cosmetic product are selected from: water; emulsifier(s), such as Laureth-4, polysorbates, and potassium cetyl sulfate; preservative(s); thickener(s), such as xanthan gum, gelatin, silica, or cetyl alcohol; emollient(s), such as waxes, oils, or silicones; pigment(s), such as iron oxide, manganese, mica flakes, or beet powder; glitter, such as mica or bismuth oxychloride; and/or fragrance(s).

In some embodiments of the invention, the cosmetic product comprises a buffer. In some embodiments, the buffer is a phosphate buffer e.g. a sodium phosphate buffer. The pH of the buffer is typically selected for compatibility with skin, e.g. pH 4 to pH 7.5; pH 5 to pH 7.5; pH 5.5 to pH 7.5; pH 6 to pH 7.5; pH 4 to pH 7; pH 5 to pH 7; pH 5.5 to pH 7; pH 6 to pH 7; pH 4 to pH 6.5; pH 5 to pH 6.5; pH 5.5 to pH 6.5; pH 6 to pH 6.5; pH 4 to pH 6; pH 5 to pH 6; or pH 5.5 to pH 6. In some embodiments, the pH of the buffer is pH 5.5 to pH 6.5, e.g. pH 5.7 to pH 6.3; or pH 5.9 to pH 6.1. In some embodiments, the cosmetic product comprises a buffer salt (e.g. sodium phosphate) concentration of 1 mM to 10 mM, e.g., 1 mM to 9 mM; 1 mM to 8 mM; 1 mM to 7 mM; 1 mM to 6 mM; 1 mM to 5 mM; 2 mM to 9 mM; 2 mM to 8 mM; 2 mM to 7 mM; 2 mM to 6 mM; 2 mM to 5 mM; 3 mM to 9 mM; 3 mM to 8 mM; 3 mM to 7 mM; 3 mM to 6 mM; 3 mM to 5 mM; 4 mM to 9 mM; 4 mM to 8 mM; 4 mM to 7 mM; 4 mM to 6 mM; or 4 mM to 5 mM. In some embodiments, the cosmetic product comprises a buffer salt (e.g. sodium phosphate) concentration of 4 mM to 6 mM, e.g. 4.5 mM to 5.5 mM; or 4.7 mM to 5.3 mM. In some embodiments, the cosmetic product comprises a sodium phosphate buffer at a concentration of 4 mM to 6 mM and a pH of pH 5.5 to pH 6.5.

In some embodiments, the composition comprises 100 μg/L to 1500 μg/L of polypeptide. In some embodiments, the composition comprises 200 μg/L to 1500 μg/L, 300 μg/L to 1500 μg/L, 400 μg/L to 1500 μg/L, 500 μg/L to 1500 g/L, 600 μg/L to 1500 μg/L, 700 μg/L to 1500 μg/L, 800 μg/L to 1500 μg/L, 900 μg/L to 1500 μg/L, 1000 μg/L to 1500 μg/L, 1100 μg/L to 1500 μg/L, 1200 μg/L to 1500 μg/L, 1300 μg/L to 1500 μg/L, 1400 μg/L to 1500 μg/L, 100 μg/L to 1400 μg/L, 100 μg/L to 1300 μg/L, 100 μg/L to 1200 g/L, 100 μg/L to 1100 g/L, 100 μg/L to 1000 μg/L, 100 μg/L to 900 μg/L, 100 μg/L to 800 μg/L, 100 g/L to 700 μg/L, 100 μg/L to 600 μg/L, 100 μg/L to 500 μg/L, 100 μg/L to 400 μg/L, 100 μg/L to 300 μg/L, or 100 μg/L to 200 μg/L of polypeptide. Wherein the composition comprises two or more polypeptides, the composition may comprise a total polypeptide concentration of 100 μg/L to 1500 μg/L. Alternatively, wherein the composition comprises two or more polypeptides, each polypeptide may be present at a concentration of 100 μg/L to 1500 μg/L. For example, the composition may comprise 100 μg/L to 500 μg/L of SOD3, 100 μg/L to 500 μg/L of SOD1, and 100 μg/L to 500 μg/L of SCF.

The advantages of the invention are not restricted to any particular type of polypeptide production method. Caviar polypeptides of the invention are typically recombinant or synthetic. Recombinant methods of protein production are well-known, and include e.g. nucleic acid expression in a recombinant host cell. Synthetic methods of protein production are also well-known, and include e.g. cell-free protein synthesis; or solid-phase chemical synthesis.

### DETAILED DESCRIPTION OF THE INVENTION

The present invention is based on the surprising discovery of key caviar polypeptides which possess highly desirable cellular functions, particularly for cosmetic applications.

In an attempt to identify the polypeptide repertoire within caviar, the inventors analysed caviar from Acipenser baerii by liquid chromatography-mass spectrometry. Significant technical challenges were encountered throughout this analysis, which required adaptation of existing mass spectrometry techniques. The adapted method included making a biphasic solution of oil and water, followed by bespoke ammonium sulphate precipitation, and urea isolation of polypeptides, followed by mass fingerprinting. Having identified the polypeptide repertoire within caviar, the inventors meticulously analysed the cellular functions associated with the identified polypeptides, and determined which of those functions is desirable in cosmetic applications (and is highly desired of caviar).

The key caviar polypeptides of the invention include:
a) a superoxide dismutase (SOD3) polypeptide;
b) a superoxide dismutase (SOD1) polypeptide;
c) a tissue inhibitor of metalloproteinase 1 (TIMP1) polypeptide;
d) an acidic fibroblast growth factor (aFGF) polypeptide;
e) a basic fibroblast growth factor (bFGF) polypeptide;
f) an insulin-like growth factor 2 (IGF-2) polypeptide; and
g) a laminin polypeptide.

In a preferred embodiment, compositions of the invention comprise one or more key caviar polypeptides of the invention and SCF. For example, compositions of the invention may comprise SOD3 and SCF; SOD1 and SCF; or SOD1, SOD3 and SCF.

### Caviar Superoxide Dismutase (SOD3)

Superoxide dismutase (SOD3) is an antioxidant enzyme that catalyzes the dismutation of two superoxide radicals into hydrogen peroxide and oxygen. Cosmetic benefits of SOD3 activity include anti-aging, promotion of hair growth, and antioxidant activity.

Prior to the present invention, the presence of SOD3 in caviar was unknown. Indeed, the corresponding NCBI reference (XP_033870136.2) predicts, by automated computational analysis, the presence of SOD3 only within the testes and blood.

A SOD3 polypeptide of the invention may be recombinant or synthetic.

In some embodiments, a SOD3 polypeptide of the invention comprises an amino acid sequence having at least 70% sequence identity to SEQ ID NO: 1. In some embodiments, the SOD3 polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the SOD3 polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to a functional fragment or catalytic domain of SEQ ID NO: 1.

In some embodiments, a SOD3 polypeptide of the invention comprises at least 50 consecutive amino acids of SEQ ID NO: 1. In some embodiments, a SOD3 polypeptide of the invention comprises at least 50, at least 60, at least 70, at least 80, at least 90, at least 100, at least 110, at least 120, at least 130, at least 140, at least 150, at least 160, at least 170, at least 180, at least 190, at least 200, at least 225, at least 240, at least 241 or at least 242 consecutive amino acids of SEQ ID NO: 1. In some embodiments, a SOD3 polypeptide of the invention comprises at least 50, at least 60, at least 70, at least 80, at least 90, at least 100, at least 110, at least 120, at least 130, at least 140, at least 150, at least 160, at least 170, at least 180, at least 190, at least 200, at least 225, at least 240, at least 241 or at least 242 consecutive amino acids of SEQ ID NO: 1, wherein the consecutive amino acids comprise the catalytic domain of the SOD3 polypeptide.

The SOD3 polypeptide sequence is represented by SEQ ID NO: 1:

(SEQ ID NO: 1)
MTMSAFSFLLALAIAGTHVSHSEESPTSEENTMKNIESKVNDLWQSLLHP
VAFVAKDAELVYASCEMKPSTKLEEGKPQVTGKVLFKQAYPQGRLESIIN
LEGFPKTSNQSRAIHIHEFGDLSDGCDAAGGHFNPFKVNHPRHPGDFGNF
LPKNSQIKTLKKNIQATMFGPNSFLSRSVVIHELKDDLGKGDNPASLLNG
NAGKRLACCVIGISNKNLWEKTSQSLTSSKKKRNARGLANKQA.

The catalytic domain of SOD3 comprises the amino acid sequence:

HFNPFKVNHPRHPGD (SEQ ID NO: 17; underlined amino acids indicate key catalytic site residues).

A SOD3 polypeptide of the invention may have one or more (e.g., up to 2, 3, 5, 10, 20, 30, 40, or 50) conservative amino acid substitutions relative to the polypeptide of SEQ ID NO: 1.

The invention also provides a fusion protein that includes at least a portion (e.g., a fragment or domain) of a SOD3 polypeptide of the invention attached to one or more fusion segments, which are typically heterologous to the SOD3 polypeptide.

A SOD3 nucleic acid of the invention encodes a SOD3 polypeptide of the invention. The SOD3 nucleic acid is typically an isolated nucleic acid. A SOD3 nucleic acid of the invention may be optimized for expression in a recombinant cell.

In some embodiments, the SOD3 nucleic acid sequence has at least 70% sequence identity to SEQ ID NO: 9. In some embodiments, the SOD3 nucleic acid sequence has at least 75%, at least 80%, at least 85%, at least 90%, at least 95%, at least 96%, at least 97%, at least 98%, or at least 99%, or 100% sequence identity to SEQ ID NO: 9, over a region of at least about 10, e.g., at least about 15, 20, 25, 30, 35, 40, 45, 50, 75, 100, 150, 200, 250, 300, 350, 400, 450, 500, 550, 600, 650, 700, or 729 nucleotides, or a catalytic domain thereof.

SEQ ID NO: 9 represents a nucleic acid sequence corresponding to the SOD3 polypeptide (SEQ ID NO: 1) with a S. cerevisiae codon bias, to optimize for expression in Pichia pastoris:

(SEQ ID NO: 9)
ATGACTATGTCTGCTTTTTCTTTTTTGTTGGCTTTGGCTATTGCTGGTAC
TCATGTTTCTCATTCTGAAGAATCTCCAACTTCTGAAGAAAATACTATGA
AAAATATTGAATCTAAAGTTAATGATTTGTGGCAATCTTTGTTGCATCCA
GTTGCTTTTGTTGCTAAAGATGCTGAATTGGTTTATGCTTCTTGTGAAAT
GAAACCATCTACTAAATTGGAAGAAGGTAAACCACAAGTTACTGGTAAAG
TTTTGTTTAAACAAGCTTATCCACAAGGTAGATTGGAATCTATTATTAAT
TTGGAAGGTTTTCCAAAAACTTCTAATCAATCTAGAGCTATTCATATTCA
TGAATTTGGTGATTTGTCTGATGGTTGTGATGCTGCTGGTGGTCATTTTA
ATCCATTTAAAGTTAATCATCCAAGACATCCAGGTGATTTTGGTAATTTT
TTGCCAAAAAATTCTCAAATTAAAACTTTGAAAAAAAATATTCAAGCTAC
TATGTTTGGTCCAAATTCTTTTTTGTCTAGATCTGTTGTTATTCATGAAT
TGAAAGATGATTTGGGTAAAGGTGATAATCCAGCTTCTTTGTTGAATGGT
AATGCTGGTAAAAGATTGGCTTGTTGTGTTATTGGTATTTCTAATAAAAA
TTTGTGGGAAAAAACTTCTCAATCTTTGACTTCTTCTAAAAAAAAAAGAA
ATGCTAGAGGTTTGGCTAATAAACAAGCT.

### Caviar Superoxide Dismutase (SOD1)

Superoxide dismutase (SOD1) is an antioxidant enzyme that catalyses the breakdown of superoxide radicals. Cosmetic benefits of SOD1 activity include anti-aging, promotion of hair growth, and antioxidant activity.

Prior to the present invention, the presence of SOD1 in caviar was unknown. Indeed, the corresponding NCBI reference (XP_033865220.2) predicts, by automated computational analysis, the presence of SOD1 only within the testes and blood.

A SOD1 polypeptide of the invention may be recombinant or synthetic. In some embodiments, a SOD1 polypeptide of the invention comprises an amino acid sequence having at least 70% sequence identity to SEQ ID NO: 2. In some embodiments, the SOD1 polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 2. In some embodiments, the SOD1 polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to a functional fragment or catalytic domain of SEQ ID NO: 2.

In some embodiments, a SOD1 polypeptide of the invention comprises at least 50 consecutive amino acids of SEQ ID NO: 2. In some embodiments, a SOD1 polypeptide of the invention comprises at least 50, at least 60, at least 70, at least 80, at least 90, at least 100, at least 110, at least 120, at least 130, at least 140, at least 150, at least 151, at least 152, at least 153 or at least 154 consecutive amino acids of SEQ ID NO: 2. In some embodiments, a SOD1 polypeptide of the invention comprises at least 50, at least 60, at least 70, at least 80, at least 90, at least 100, at least 110, at least 120, at least 130, at least 140, at least 150, at least 151, at least 152, at least 153 or at least 154 consecutive amino acids of SEQ ID NO: 2, wherein the consecutive amino acids comprise the catalytic domain of the SOD1 polypeptide.

The SOD1 polypeptide sequence is represented by SEQ ID NO: 2:

(SEQ ID NO: 2)
MVLKAVCVLKGTGDVCGTVHFVQEKEAGPVKLTGQITGLTPGEHGFHVHA
FGDNTNGCASAGPHFNPLGKTHGAPQDEIRHIGDLGNVIAGDDKVAIINI
EDKLITLSGAYSIIGRTMVIHEKADDLGKGGNDESLVTGNAGGRLACGVI
GIAQS.

The catalytic domain of SOD1 comprises the amino acid sequence:

GFHVHAFGDNT (SEQ ID NO: 18; underlined amino acids indicate key catalytic site residues)

A SOD1 polypeptide of the invention may have one or more (e.g., up to 2, 3, 5, 10, 20, 30, 40, or 50) conservative amino acid substitutions relative to the polypeptide of SEQ ID NO: 2.

The invention also provides a fusion protein that includes at least a portion (e.g., a fragment or domain) of a SOD1 polypeptide of the invention attached to one or more fusion segments, which are typically heterologous to the SOD1 polypeptide.

A SOD1 nucleic acid of the invention encodes a SOD1 polypeptide of the invention. The SOD1 nucleic acid is typically an isolated nucleic acid. A SOD1 nucleic acid of the invention may be optimized for expression in a recombinant cell.

In some embodiments, the SOD1 nucleic acid sequence has at least 70% sequence identity to SEQ ID NO: 10. In some embodiments, the SOD1 nucleic acid sequence has at least 75%, at least 80%, at least 85%, at least 90%, at least 95%, at least 96%, at least 97%, at least 98%, or at least 99%, or 100% sequence identity to SEQ ID NO: 10, over a region of at least about 10, e.g., at least about 15, 20, 25, 30, 35, 40, 45, 50, 75, 100, 150, 200, 250, 300, 350, 400, 450, or 465 nucleotides, or a catalytic domain thereof.

SEQ ID NO: 10 represents a nucleic acid sequence corresponding to the SOD1 polypeptide (SEQ ID NO: 2) with a S. cerevisiae codon bias, to optimize for expression in Pichia pastoris:

(SEQ ID NO: 10)
ATGGTTTTGAAAGCTGTTTGTGTTTTGAAAGGTACTGGTGATGTTTGTGG
TACTGTTCATTTTGTTCAAGAAAAAGAAGCTGGTCCAGTTAAATTGACTG
GTCAAATTACTGGTTTGACTCCAGGTGAACATGGTTTTCATGTTCATGCT
TTTGGTGATAATACTAATGGTTGTGCTTCTGCTGGTCCACATTTTAATCC
ATTGGGTAAAACTCATGGTGCTCCACAAGATGAAATTAGACATATTGGTG
ATTTGGGTAATGTTATTGCTGGTGATGATAAAGTTGCTATTATTAATATT
GAAGATAAATTGATTACTTTGTCTGGTGCTTATTCTATTATTGGTAGAAC
TATGGTTATTCATGAAAAAGCTGATGATTTGGGTAAAGGTGGTAATGATG
AATCTTTGGTTACTGGTAATGCTGGTGGTAGATTGGCTTGTGGTGTTATT
GGTATTGCTCAATCT.

### Caviar Tissue Inhibitor of Metalloproteinase 1 (TIMP1)

Tissue inhibitor of metalloproteinase 1 (TIMP1) is involved in the degradation of extracellular matrix, is able to promote cell proliferation in a wide range of cell types, and is believed to have an anti-apoptotic function. Cosmetic benefits of TIMP1 activity include anti-aging, promotion of hair growth, and wound healing.

Prior to the present invention, the presence of TIMP1 in caviar was unknown. Indeed, the corresponding NCBI reference (XP_034775618.1) predicts, by automated computational analysis, the presence of TIMP1 only within the testes and blood.

A TIMP1 polypeptide of the invention may be recombinant or synthetic. In some embodiments, a TIMP1 polypeptide of the invention comprises an amino acid sequence having at least 70% sequence identity to SEQ ID NO: 3. In some embodiments, the TIMP1 polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 3. In some embodiments, the TIMP1 polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to a functional fragment or catalytic domain of SEQ ID NO: 3.

In some embodiments, a TIMP1 polypeptide of the invention comprises at least 50 consecutive amino acids of SEQ ID NO: 3. In some embodiments, a TIMP1 polypeptide of the invention comprises at least 50, at least 60, at least 70, at least 80, at least 90, at least 100, at least 110, at least 120, at least 130, at least 140, at least 150, at least 160, at least 170, at least 180, at least 190, at least 200, at least 204, at least 205 or at least 206 consecutive amino acids of SEQ ID NO: 3. In some embodiments, a TIMP1 polypeptide of the invention comprises at least 50, at least 60, at least 70, at least 80, at least 90, at least 100, at least 110, at least 120, at least 130, at least 140, at least 150, at least 160, at least 170, at least 180, at least 190, at least 200, at least 204, at least 205 or at least 206 consecutive amino acids of SEQ ID NO: 3, wherein the consecutive amino acids comprise the catalytic domain of the TIMP1 polypeptide.

The TIMP1 polypeptide sequence is represented by SEQ ID NO: 3:

(SEQ ID NO: 3)
MAPLAPLASCILLLLWLAAPSRACTCAPLHPQTAFCSSDFIIRAKFVG
TAEVNQTALSQRYEIKMTKMFKGFSALGDASDIRFVYTPTAESVCGYF
HRSQNRSEEFLIAGKLRNGHLHINTCSYVVPWNSLSSSQRRGFTKTYA
AGCEECTVFSCSSIPCKLQNDTHCLWTDQFLTGTDKGFQSRHLACLPR
EPGICTWQSLRTRMA.

A TIMP1 polypeptide of the invention may have one or more (e.g., up to 2, 3, 5, 10, 20, 30, 40, or 50) conservative amino acid substitutions relative to the polypeptide of SEQ ID NO: 3.

The invention also provides a fusion protein that includes at least a portion (e.g., a fragment or domain) of a TIMP1 polypeptide of the invention attached to one or more fusion segments, which are typically heterologous to the TIMP1 polypeptide.

A TIMP1 nucleic acid of the invention encodes a TIMP1 polypeptide of the invention. The TIMP1 nucleic acid is typically an isolated nucleic acid. A TIMP1 nucleic acid of the invention may be optimized for expression in a recombinant cell.

In some embodiments, the TIMP1 nucleic acid sequence has at least 70% sequence identity to SEQ ID NO: 11. In some embodiments, the TIMP1 nucleic acid sequence has at least 75%, at least 80%, at least 85%, at least 90%, at least 95%, at least 96%, at least 97%, at least 98%, or at least 99%, or 100% sequence identity to SEQ ID NO: 11, over a region of at least about 10, e.g., at least about 15, 20, 25, 30, 35, 40, 45, 50, 75, 100, 150, 200, 250, 300, 350, 400, 450, 500, 550, 600, or 621 nucleotides, or a catalytic domain thereof.

SEQ ID NO: 11 represents a nucleic acid sequence corresponding to the TIMP1 polypeptide (SEQ ID NO: 3) with a S. cerevisiae codon bias, to optimize for expression in Pichia pastoris:

(SEQ ID NO: 11)
ATGGCTCCATTGGCTCCATTGGCTTCTTGTATTTTGTTGTTGTTGTGG
TTGGCTGCTCCATCTAGAGCTTGTACTTGTGCTCCATTGCATCCACAA
ACTGCTTTTTGTTCTTCTGATTTTATTATTAGAGCTAAATTTGTTGGT
ACTGCTGAAGTTAATCAAACTGCTTTGTCTCAAAGATATGAAATTAAA
ATGACTAAAATGTTTAAAGGTTTTTCTGCTTTGGGTGATGCTTCTGAT
ATTAGATTTGTTTATACTCCAACTGCTGAATCTGTTTGTGGTTATTTT
CATAGATCTCAAAATAGATCTGAAGAATTTTTGATTGCTGGTAAATTG
AGAAATGGTCATTTGCATATTAATACTTGTTCTTATGTTGTTCCATGG
AATTCTTTGTCTTCTTCTCAAAGAAGAGGTTTTACTAAAACTTATGCT
GCTGGTTGTGAAGAATGTACTGTTTTTTCTTGTTCTTCTATTCCATGT
AAATTGCAAAATGATACTCATTGTTTGTGGACTGATCAATTTTTGACT
GGTACTGATAAAGGTTTTCAATCTAGACATTTGGCTTGTTTGCCAAGA
GAACCAGGTATTTGTACTTGGCAATCTTTGAGAACTAGAATGGCT.

Caviar Acidic Fibroblast Growth Factor (aFGF)

Acidic fibroblast growth factor (aFGF), also known as fibroblast growth factor 1, is a fibroblast activator protein. Cosmetic benefits of aFGF activity include anti-aging, anti-wrinkle, wound-healing, anti-hair loss and promotion of hair growth.

Prior to the present invention, the presence of aFGF in caviar was unknown. Indeed, the corresponding GenBank reference (RXM90924.1) predicts, by automated computational analysis, the presence of aFGF only within the blood.

An aFGF polypeptide of the invention may be recombinant or synthetic. In some embodiments, an aFGF polypeptide of the invention comprises an amino acid sequence having at least 70% sequence identity to SEQ ID NO: 4. In some embodiments, the aFGF polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 4. In some embodiments, the aFGF polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to a functional fragment or catalytic domain of SEQ ID NO: 4.

In some embodiments, an aFGF polypeptide of the invention comprises at least 50 consecutive amino acids of SEQ ID NO: 4. In some embodiments, an aFGF polypeptide of the invention comprises at least 50, at least 60, at least 70, at least 80, at least 90, at least 100, at least 110, at least 120, at least 130, at least 140, at least 150, at least 151, at least 152, at least 153 or at least 154 consecutive amino acids of SEQ ID NO: 4 . . . . In some embodiments, an aFGF polypeptide of the invention comprises at least 50, at least 60, at least 70, at least 80, at least 90, at least 100, at least 110, at least 120, at least 130, at least 140, at least 150, at least 151, at least 152, at least 153 or at least 154 consecutive amino acids of SEQ ID NO: 4), wherein the consecutive amino acids comprise the catalytic domain of the aFGF polypeptide.

The aFGF polypeptide sequence is represented by SEQ ID NO: 4:

(SEQ ID NO: 4)
MAEGKVTVLTILPEKFNLHLENYKKPNLLYCYNGGYFLRVLPNGAVDG
IRDRSDKHIQLQVTAENVGVVSIKGLEAGRYLAMSTDGQLCGSQTLTD
ECFFLETLEENHYATYKAQKYQDRNWYVGIKKNGRCKSGEKTHIGQKA
ILFLPLSASSD.

An aFGF polypeptide of the invention may have one or more (e.g., up to 2, 3, 5, 10, 20, 30, 40, or 50) conservative amino acid substitutions relative to the polypeptide of SEQ ID NO: 4.

The invention also provides a fusion protein that includes at least a portion (e.g., a fragment or domain) of an aFGF polypeptide of the invention attached to one or more fusion segments, which are typically heterologous to the aFGF polypeptide.

An aFGF nucleic acid of the invention encodes an aFGF polypeptide of the invention. The aFGF nucleic acid is typically an isolated nucleic acid. An aFGF nucleic acid of the invention may be optimized for expression in a recombinant cell.

In some embodiments, the aFGF nucleic acid sequence has at least 70% sequence identity to SEQ ID NO: 13. In some embodiments, the aFGF nucleic acid sequence has at least 75%, at least 80%, at least 85%, at least 90%, at least 95%, at least 96%, at least 97%, at least 98%, or at least 99%, or 100% sequence identity to SEQ ID NO: 13, over a region of at least about 10, e.g., at least about 15, 20, 25, 30, 35, 40, 45, 50, 75, 100, 150, 200, 250, 300, 350, 400, 450, or 465 nucleotides, or a catalytic domain thereof.

SEQ ID NO: 13 represents a nucleic acid sequence corresponding to the aFGF polypeptide (SEQ ID NO: 1) with a S. cerevisiae codon bias, to optimize for expression in Pichia pastoris:

(SEQ ID NO: 13)
ATGGCTGAAGGTAAAGTTACTGTTTTGACTATTTTGCCAGAAAAATTT
AATTTGCATTTGGAAAATTATAAAAAACCAAATTTGTTGTATTGTTAT
AATGGTGGTTATTTTTTGAGAGTTTTGCCAAATGGTGCTGTTGATGGT
ATTAGAGATAGATCTGATAAACATATTCAATTGCAAGTTACTGCTGAA
AATGTTGGTGTTGTTTCTATTAAAGGTTTGGAAGCTGGTAGATATTTG
GCTATGTCTACTGATGGTCAATTGTGTGGTTCTCAAACTTTGACTGAT
GAATGTTTTTTTTTGGAAACTTTGGAAGAAAATCATTATGCTACTTAT
AAAGCTCAAAAATATCAAGATAGAAATTGGTATGTTGGTATTAAAAAA
AATGGTAGATGTAAATCTGGTGAAAAAACTCATATTGGTCAAAAAGCT
ATTTTGTTTTTGCCATTGTCTGCTTCTTCTGAT.

Caviar Basic Fibroblast Growth Factor (bFGF)

Basic fibroblast growth factor (bFGF), also known as fibroblast growth factor 2, is a fibroblast activator protein. Cosmetic benefits of bFGF activity include anti-aging, anti-wrinkle, wound-healing, anti-hair loss and promotion of hair growth.

Prior to the present invention, the presence of bFGF in caviar was unknown. Indeed, the corresponding GenBank reference (RXM97423.1) predicts, by automated computational analysis, the presence of bFGF only within the blood.

A bFGF polypeptide of the invention may be recombinant or synthetic. In some embodiments, a bFGF polypeptide of the invention comprises an amino acid sequence having at least 70% sequence identity to SEQ ID NO: 5. In some embodiments, the bFGF polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 5. In some embodiments, the bFGF polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to a functional fragment or catalytic domain of SEQ ID NO: 5.

In some embodiments, a bFGF polypeptide of the invention comprises at least 50 consecutive amino acids of SEQ ID NO: 5. In some embodiments, a bFGF polypeptide of the invention comprises at least 50, at least 60, at least 70, at least 80, at least 90, at least 100, at least 110, at least 120, at least 130, at least 140, at least 150, at least 151, at least 152, at least 153 or at least 154 consecutive amino acids of SEQ ID NO: 5. In some embodiments, a bFGF polypeptide of the invention comprises at least 50, at least 60, at least 70, at least 80, at least 90, at least 100, at least 110, at least 120, at least 130, at least 140, at least 150, at least 151, at least 152, at least 153 or at least 154 consecutive amino acids of SEQ ID NO: 5, wherein the consecutive amino acids comprise the catalytic domain of the bFGF polypeptide.

The bFGF polypeptide sequence is represented by SEQ ID NO: 5:

(SEQ ID NO: 5)
MAAGGITTFPTVPDDGGSSTFPPGNFKEPKRLYCKNGGYFLRINPDGR
VDGIREKDDPRIKLQLQAESIGVVSIKGVSANRYLAMNEDGRLFGSKC
TTDECFFFERLESNNYNTYRSQKYPDWYVALKRTGQFKSGSKTGPGQK
AILFLPMSAKS.

A bFGF polypeptide of the invention may have one or more (e.g., up to 2, 3, 5, 10, 20, 30, 40, or 50) conservative amino acid substitutions relative to the polypeptide of SEQ ID NO: 5.

The invention also provides a fusion protein that includes at least a portion (e.g., a fragment or domain) of a bFGF polypeptide of the invention attached to one or more fusion segments, which are typically heterologous to the bFGF polypeptide.

A bFGF nucleic acid of the invention encodes a bFGF polypeptide of the invention. The bFGF nucleic acid is typically an isolated nucleic acid. A bFGF nucleic acid of the invention may be optimized for expression in a recombinant cell.

In some embodiments, the bFGF nucleic acid sequence has at least 70% sequence identity to SEQ ID NO: 14. In some embodiments, the bFGF nucleic acid sequence has at least 75%, at least 80%, at least 85%, at least 90%, at least 95%, at least 96%, at least 97%, at least 98%, or at least 99%, or 100% sequence identity to SEQ ID NO: 14, over a region of at least about 10, e.g., at least about 15, 20, 25, 30, 35, 40, 45, 50, 75, 100, 150, 200, 250, 300, 350, 400, 450, or 465 nucleotides, or a catalytic domain thereof.

SEQ ID NO: 14 represents a nucleic acid sequence corresponding to the bFGF polypeptide (SEQ ID NO: 5) with a S. cerevisiae codon bias, to optimize for expression in Pichia pastoris:

(SEQ ID NO: 14)
ATGGCTGCTGGTGGTATTACTACTTTTCCAACTGTTCCAGATGATGGT
GGTTCTTCTACTTTTCCACCAGGTAATTTTAAAGAACCAAAAAGATTG
TATTGTAAAAATGGTGGTTATTTTTTGAGAATTAATCCAGATGGTAGA
GTTGATGGTATTAGAGAAAAAGATGATCCAAGAATTAAATTGCAATTG
CAAGCTGAATCTATTGGTGTTGTTTCTATTAAAGGTGTTTCTGCTAAT
AGATATTTGGCTATGAATGAAGATGGTAGATTGTTTGGTTCTAAATGT
ACTACTGATGAATGTTTTTTTTTTGAAAGATTGGAATCTAATAATTAT
AATACTTATAGATCTCAAAAATATCCAGATTGGTATGTTGCTTTGAAA
AGAACTGGTCAATTTAAATCTGGTTCTAAAACTGGTCCAGGTCAAAAA
GCTATTTTGTTTTTGCCAATGTCTGCTAAATCT.

### Caviar Insulin-Like Growth Factor 2 (IGF-2)

Insulin-like growth factor 2 (IGF-2) promotes growth and proliferation of cells. Cosmetic benefits of IGF-2 activity include anti-aging, anti-wrinkle, wound-healing, anti-hair loss and promotion of hair growth.

Prior to the present invention, the presence of IGF-2 in caviar was unknown. Indeed, the corresponding GenBank reference (RXM35556.1) predicts, by automated computational analysis, the presence of IGF-2 only within the blood.

An IGF-2 polypeptide of the invention may be recombinant or synthetic. In some embodiments, an IGF-2 polypeptide of the invention comprises an amino acid sequence having at least 70% sequence identity to SEQ ID NO: 6. In some embodiments, the IGF-2 polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 6. In some embodiments, the IGF-2 polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to a functional fragment or catalytic domain of SEQ ID NO: 6.

In some embodiments, an IGF-2 polypeptide of the invention comprises at least 50 consecutive amino acids of SEQ ID NO: 6. In some embodiments, an IGF-2 polypeptide of the invention comprises at least 50, at least 60, at least 70, at least 80, at least 90, at least 100, at least 110, at least 120, at least 130, at least 140, at least 150, at least 160, at least 170, at least 180, at least 190, at least 200, at least 210, at least 215, at least 216, at least 217, or at least 218 consecutive amino acids of SEQ ID NO: 6. In some embodiments, an IGF-2 polypeptide of the invention comprises at least 50, at least 60, at least 70, at least 80, at least 90, at least 100, at least 110, at least 120, at least 130, at least 140, at least 150, at least 160, at least 170, at least 180, at least 190, at least 200, at least 210, at least 215, at least 216, at least 217, or at least 218 consecutive amino acids of SEQ ID NO: 6, wherein the consecutive amino acids comprise the catalytic domain of the IGF-2 polypeptide.

The IGF-2 polypeptide sequence is represented by SEQ ID NO: 6:

(SEQ ID NO: 6)
MEDHLSYNKHQAYCHNCIESGNSSNSIKVRKMSTSRQLLVFTIALTVY
IVDVANSIASAETLCGGELVDTLQFVCGDRGFYFSKPPSRSNIRRSQK
GIVEVCCYSSCDLRLLEMYCAKPAKSERDVSSTQFQVIPLALNKDVSK
KPIIAKYSKYELWQKKAAQRLRRGVPSILKARKFRRHAEEIKALEQTR
FHRPLITLPSKQPVTVKPLTENYASKK.

An IGF-2 polypeptide of the invention may have one or more (e.g., up to 2, 3, 5, 10, 20, 30, 40, or 50) conservative amino acid substitutions relative to the polypeptide of SEQ ID NO: 15.

The invention also provides a fusion protein that includes at least a portion (e.g., a fragment or domain) of an IGF-2 polypeptide of the invention attached to one or more fusion segments, which are typically heterologous to the IGF-2 polypeptide.

An IGF-2 nucleic acid of the invention encodes an IGF-2 polypeptide of the invention. The IGF-2 nucleic acid is typically an isolated nucleic acid. An IGF-2 nucleic acid of the invention may be optimized for expression in a recombinant cell.

In some embodiments, the IGF-2 nucleic acid sequence has at least 70% sequence identity to SEQ ID NO: 15. In some embodiments, the IGF-2 nucleic acid sequence has at least 75%, at least 80%, at least 85%, at least 90%, at least 95%, at least 96%, at least 97%, at least 98%, or at least 99%, or 100% sequence identity to SEQ ID NO: 15, over a region of at least about 10, e.g., at least about 15, 20, 25, 30, 35, 40, 45, 50, 75, 100, 150, 200, 250, 300, 350, 400, 450, 500, 550, 600, 650, or 657 nucleotides, or a catalytic domain thereof.

SEQ ID NO: 15 represents a nucleic acid sequence corresponding to the IGF-2 polypeptide (SEQ ID NO: 6) with a S. cerevisiae codon bias, to optimize for expression in Pichia pastoris:

(SEQ ID NO: 15)
ATGGAAGATCATTTGTCTTATAATAAACATCAAGCTTATTGTCATAAT
TGTATTGAATCTGGTAATTCTTCTAATTCTATTAAAGTTAGAAAAATG
TCTACTTCTAGACAATTGTTGGTTTTTACTATTGCTTTGACTGTTTAT
ATTGTTGATGTTGCTAATTCTATTGCTTCTGCTGAAACTTTGTGTGGT
GGTGAATTGGTTGATACTTTGCAATTTGTTTGTGGTGATAGAGGTTTT
TATTTTTCTAAACCACCATCTAGATCTAATATTAGAAGATCTCAAAAA
GGTATTGTTGAAGTTTGTTGTTATTCTTCTTGTGATTTGAGATTGTTG
GAAATGTATTGTGCTAAACCAGCTAAATCTGAAAGAGATGTTTCTTCT
ACTCAATTTCAAGTTATTCCATTGGCTTTGAATAAAGATGTTTCTAAA
AAACCAATTATTGCTAAATATTCTAAATATGAATTGTGGCAAAAAAAA
GCTGCTCAAAGATTGAGAAGAGGTGTTCCATCTATTTTGAAAGCTAGA
AAATTTAGAAGACATGCTGAAGAAATTAAAGCTTTGGAACAAACTAGA
TTTCATAGACCATTGATTACTTTGCCATCTAAACAACCAGTTACTGTT
AAACCATTGACTGAAAATTATGCTTCTAAAAAA.

### Caviar Laminin

Laminin is an important and biologically active part of the basal lamina, influencing cell differentiation, migration and adhesion. Cosmetic benefits of laminin activity include anti-aging and hair growth.

Prior to the present invention, the presence of laminin in caviar was unknown. Indeed, the corresponding GenBank reference (RXN01125.1) predicts, by automated computational analysis, the presence of laminin only within the blood.

A laminin polypeptide of the invention may be recombinant or synthetic. In some embodiments, a laminin polypeptide of the invention comprises an amino acid sequence having at least 70% sequence identity to SEQ ID NO: 7. In some embodiments, the laminin polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 7. In some embodiments, the laminin polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to a functional fragment or catalytic domain of SEQ ID NO: 7.

In some embodiments, a laminin polypeptide of the invention comprises at least 50 consecutive amino acids of SEQ ID NO: 7. In some embodiments, a laminin polypeptide of the invention comprises at least 50, at least 60, at least 70, at least 80, at least 90, at least 100, at least 110, at least 120, at least 130, at least 140, at least 150, at least 160, at least 170, at least 180, at least 190, at least 200, at least 225, at least 250, at least 275, at least 300, at least 350, at least 400, at least 450, at least 500, at least 600, at least 700, at least 800, at least 900, at least 1000, at least 1250, at least 1500, at least 1750, at least 2000, at least 2250, at least 2280, at least 2281, at least 2282, at least 2283, or at least 2284 consecutive amino acids of SEQ ID NO: 7. In some embodiments, a laminin polypeptide of the invention comprises at least 50, at least 60, at least 70, at least 80, at least 90, at least 100, at least 110, at least 120, at least 130, at least 140, at least 150, at least 160, at least 170, at least 180, at least 190, at least 200, at least 225, at least 250, at least 275, at least 300, at least 350, at least 400, at least 450, at least 500, at least 600, at least 700, at least 800, at least 900, at least 1000, at least 1250, at least 1500, at least 1750, at least 2000, at least 2250, at least 2280, at least 2281, at least 2282, at least 2283, or at least 2284 consecutive amino acids of SEQ ID NO: 7.

The laminin polypeptide sequence is represented by SEQ ID NO: 7:

(SEQ ID NO: 7)
MKIYLSLIVLMICASSVECNQRGLFPAILNLASNAEITANASCGETGP
EMFCKLVEHVPGRPIRNPQCRVCDSTSPNPRERHPISNAIDGTNNWWQ
SPSIKNGRQNHWVTITLDLRQVFQVAYIIIKAANSPRPGNWILERSID
GIEFQPWQYHAISDTECLTRYNVTPRIGPPTYKRDDEVICTSYYSRLV
PLEHGEIHTSLINGRPSADDLTPALLEFTSARYIRLRLQRIRTLNADL
MTLSYRDPKDVDPIVTRRKLQCVCEHNTCGESCNKCCPGYHQKPWKPG
TLSVGNTCEKCNCHNKTEDCYYNQTVASSMMSMNIDGQFVGGGVCIDC
TQNTAGINCETCVDGNYRPHKVSPYDENPCVECGCDLNGSQHPVCIKD
DNHADLQNGLLPGKCHCKEGYAGEKCDRCSFGYSGYPYCVKCNCSLIG
SIHFDPCVEQCTCKENVMGDNCDLCKRGYYNLQKSSPEGCTECFCFGV
SDVCESISWPLTQVLNIDDWLMPVRPIPITSGTRFDHDDSNHISKESR
ILPPQSSWSAPESFLGNKLTAYGGYLNYTLSYDVPVESLHLQLMSNFD
VVIEGNGKTLHTRFNAQLLLQPYKEQTVAVEMLPRNFMDFYTSRAIER
DRLMTVLANLTPTVNAIDLKPAMDVEHCECPWGYTGTSCESCSPGHYR
VDGILFGGICRLCECNGHATECDIHGVCSDCKHNTIGPHCEQCMPGFY
GDPSDGMPDDCQVCACPLNIPSNNFSSTCHLDSAGEVMCDQCVPGYEG
PRCERCANGYYGDPTVPGELCVPCDCNGNVNPLEPGHCDTRTGECLKC
VENTAGSHCERCADGYFGDAIVAKNCQDCGCHVNSSYSSVCDLESGKC
KCRPNVTGEKCDQCMPGHFGLSSGLGCQTCNCNLAGSLSDTCDDEGQC
PCVPGVAGEKCDRCARGFYDFQDDGCTPCDCAHTHNNCNTETGQCICP
PHTAGVKCELCEANHWGHDSKLGCKPCDCSLVGSSSSQCDLSSGWCQC
NSEFGAEKCNECALGFRAYPECTACNCSIIGTREEYCDEKQGVCGCDK
DSSTCSCKVNVVGPGCDECKLGTFALSADDPLGCSPCFCFGVSETCEE
LGGLVRIPIILTPDQETLHVVSQSDLSGTLDGVFLQSPDILLDAALVK
QSLHTEPFYWRLPEQFQGNKLLAYGGKLRYTAAFYALEGSGISNFEPQ
LLIKGGRTSKLNIYRDVPAPDNGVETSQEIDLKEREWKYFNSVSDQAV
SHSDFMSVLSNIQYILIKASYGSDLQQSRISNISLEIAVDSDEMNAGR
ETARGIERCKCPSGYAGLSCQECAPGFYRQSLTELNVRGPRPLIEPSC
NDNCTGVLLNDLENLELSMQSVNLTGVILAPYSLLASLENTTDEIKML
LSPERNSSFLLKKAEEQLSGLTKDIDQLHQKTTQAYGDGQDLNKSTER
MVNQSQELLDSITKVQTAIQALVELGSNLNNTLGNNLVLTNSTQLLDE
VSAVLEKMRNKAFTQQHQNATHEFKAAEALLLQVQREFQKPQKDVMEL
KERIVTILSEHSSKLQDAQNLVNESFTKANETNHILHAMSSNLAVFYD
KKQNVSSNHFLTTEMIEEAQATMTEGLNIATDVVNATSQLEGHKDELA
LWNPKLRKQVDNLVMQMTKRSVLDLVYKAEDHAAGLKKLADSLHNVLS
GVRNVSFNATNAAQANSNIKINIEKAERLAEQANSTVAAAWSLALFAD
HSLKDAGAKSLQQSSRLLSEAADLNNKTNGLISDLNGLKEQVDKIRYN
AQNISKQLSEPLQLLSSIPNNVSVKVLEAKEHAMGANVSAVAALQHLE
DFSQKLEESSSAMIRANETVKRTNELFSDSAKTANAAEKKVQEVETQT
NLLFERLKPLKMLEDTLSRNLSEIKELIDQARKQAASIKVAVAAEQDC
VRAYKPDISSSNYNTLTLIVKTSESDNLLFYLGSSTNVDFMALEMRRG
KVSFLWDVGSGFAKLEYPDIQINNDKWHRIHATRFGKTGSLTVQELKS
SQTPTVKTATSPGTSTVLDVNKSTLVFVGGLGGQIKKSSAVKMTHFKG
CMGGASLNGNNIGLWNYAEREGKCRGCFMSPQDEDTSFHFDGSGYSVV
EKALRSTVTQIVMLFKTFSPNGLLLYLASNGTRDFTSIELVDGKVRLT
FELGSGPLSLITDKTYNSGNWYKIAFHRTKQKGYLAVMDAYIPTNRET
KEGTTSGSASDLNRSDKDPIYIGGLPRSRPVRRQLVTRSYVGCIKNLE
IARSNFDLLKESYGVKKGCVLEHGTEPWY.

A laminin polypeptide of the invention may have one or more (e.g., up to 2, 3, 5, 10, 20, 30, 40, or 50) conservative amino acid substitutions relative to the polypeptide of SEQ ID NO: 7.

The invention also provides a fusion protein that includes at least a portion (e.g., a fragment or domain) of a laminin polypeptide of the invention attached to one or more fusion segments, which are typically heterologous to the laminin polypeptide.

A laminin nucleic acid of the invention encodes a laminin polypeptide of the invention. The laminin nucleic acid is typically an isolated nucleic acid. A laminin nucleic acid of the invention may be optimized for expression in a recombinant cell.

In some embodiments, the laminin nucleic acid sequence has at least 70% sequence identity to SEQ ID NO: 16. In some embodiments, the laminin nucleic acid sequence has at least 75%, at least 80%, at least 85%, at least 90%, at least 95%, at least 96%, at least 97%, at least 98%, or at least 99%, or 100% sequence identity to SEQ ID NO: 16, over a region of at least about 10, e.g., at least about 15, 20, 25, 30, 35, 40, 45, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000, 5500, 6000, 6500 or 6855 nucleotides.

SEQ ID NO: 16 represents a nucleic acid sequence corresponding to the laminin polypeptide (SEQ ID NO: 7) with a S. cerevisiae codon bias, to optimize for expression in Pichia pastoris:

(SEQ ID NO: 16)
ATGAAAATTTATTTGTCTTTGATTGTTTTGATGATTTGTGCTTCTTCT
GTTGAATGTAATCAAAGAGGTTTGTTTCCAGCTATTTTGAATTTGGCT
TCTAATGCTGAAATTACTGCTAATGCTTCTTGTGGTGAAACTGGTCCA
GAAATGTTTTGTAAATTGGTTGAACATGTTCCAGGTAGACCAATTAGA
AATCCACAATGTAGAGTTTGTGATTCTACTTCTCCAAATCCAAGAGAA
AGACATCCAATTTCTAATGCTATTGATGGTACTAATAATTGGTGGCAA
TCTCCATCTATTAAAAATGGTAGACAAAATCATTGGGTTACTATTACT
TTGGATTTGAGACAAGTTTTTCAAGTTGCTTATATTATTATTAAAGCT
GCTAATTCTCCAAGACCAGGTAATTGGATTTTGGAAAGATCTATTGAT
GGTATTGAATTTCAACCATGGCAATATCATGCTATTTCTGATACTGAA
TGTTTGACTAGATATAATGTTACTCCAAGAATTGGTCCACCAACTTAT
AAAAGAGATGATGAAGTTATTTGTACTTCTTATTATTCTAGATTGGTT
CCATTGGAACATGGTGAAATTCATACTTCTTTGATTAATGGTAGACCA
TCTGCTGATGATTTGACTCCAGCTTTGTTGGAATTTACTTCTGCTAGA
TATATTAGATTGAGATTGCAAAGAATTAGAACTTTGAATGCTGATTTG
ATGACTTTGTCTTATAGAGATCCAAAAGATGTTGATCCAATTGTTACT
AGAAGAAAATTGCAATGTGTTTGTGAACATAATACTTGTGGTGAATCT
TGTAATAAATGTTGTCCAGGTTATCATCAAAAACCATGGAAACCAGGT
ACTTTGTCTGTTGGTAATACTTGTGAAAAATGTAATTGTCATAATAAA
ACTGAAGATTGTTATTATAATCAAACTGTTGCTTCTTCTATGATGTCT
ATGAATATTGATGGTCAATTTGTTGGTGGTGGTGTTTGTATTGATTGT
ACTCAAAATACTGCTGGTATTAATTGTGAAACTTGTGTTGATGGTAAT
TATAGACCACATAAAGTTTCTCCATATGATGAAAATCCATGTGTTGAA
TGTGGTTGTGATTTGAATGGTTCTCAACATCCAGTTTGTATTAAAGAT
GATAATCATGCTGATTTGCAAAATGGTTTGTTGCCAGGTAAATGTCAT
TGTAAAGAAGGTTATGCTGGTGAAAAATGTGATAGATGTTCTTTTGGT
TATTCTGGTTATCCATATTGTGTTAAATGTAATTGTTCTTTGATTGGT
TCTATTCATTTTGATCCATGTGTTGAACAATGTACTTGTAAAGAAAAT
GTTATGGGTGATAATTGTGATTTGTGTAAAAGAGGTTATTATAATTTG
CAAAAATCTTCTCCAGAAGGTTGTACTGAATGTTTTTGTTTTGGTGTT
TCTGATGTTTGTGAATCTATTTCTTGGCCATTGACTCAAGTTTTGAAT
ATTGATGATTGGTTGATGCCAGTTAGACCAATTCCAATTACTTCTGGT
ACTAGATTTGATCATGATGATTCTAATCATATTTCTAAAGAATCTAGA
ATTTTGCCACCACAATCTTCTTGGTCTGCTCCAGAATCTTTTTTGGGT
AATAAATTGACTGCTTATGGTGGTTATTTGAATTATACTTTGTCTTAT
GATGTTCCAGTTGAATCTTTGCATTTGCAATTGATGTCTAATTTTGAT
GTTGTTATTGAAGGTAATGGTAAAACTTTGCATACTAGATTTAATGCT
CAATTGTTGTTGCAACCATATAAAGAACAAACTGTTGCTGTTGAAATG
TTGCCAAGAAATTTTATGGATTTTTATACTTCTAGAGCTATTGAAAGA
GATAGATTGATGACTGTTTTGGCTAATTTGACTCCAACTGTTAATGCT
ATTGATTTGAAACCAGCTATGGATGTTGAACATTGTGAATGTCCATGG
GGTTATACTGGTACTTCTTGTGAATCTTGTTCTCCAGGTCATTATAGA
GTTGATGGTATTTTGTTTGGTGGTATTTGTAGATTGTGTGAATGTAAT
GGTCATGCTACTGAATGTGATATTCATGGTGTTTGTTCTGATTGTAAA
CATAATACTATTGGTCCACATTGTGAACAATGTATGCCAGGTTTTTAT
GGTGATCCATCTGATGGTATGCCAGATGATTGTCAAGTTTGTGCTTGT
CCATTGAATATTCCATCTAATAATTTTTCTTCTACTTGTCATTTGGAT
TCTGCTGGTGAAGTTATGTGTGATCAATGTGTTCCAGGTTATGAAGGT
CCAAGATGTGAAAGATGTGCTAATGGTTATTATGGTGATCCAACTGTT
CCAGGTGAATTGTGTGTTCCATGTGATTGTAATGGTAATGTTAATCCA
TTGGAACCAGGTCATTGTGATACTAGAACTGGTGAATGTTTGAAATGT
GTTGAAAATACTGCTGGTTCTCATTGTGAAAGATGTGCTGATGGTTAT
TTTGGTGATGCTATTGTTGCTAAAAATTGTCAAGATTGTGGTTGTCAT
GTTAATTCTTCTTATTCTTCTGTTTGTGATTTGGAATCTGGTAAATGT
AAATGTAGACCAAATGTTACTGGTGAAAAATGTGATCAATGTATGCCA
GGTCATTTTGGTTTGTCTTCTGGTTTGGGTTGTCAAACTTGTAATTGT
AATTTGGCTGGTTCTTTGTCTGATACTTGTGATGATGAAGGTCAATGT
CCATGTGTTCCAGGTGTTGCTGGTGAAAAATGTGATAGATGTGCTAGA
GGTTTTTATGATTTTCAAGATGATGGTTGTACTCCATGTGATTGTGCT
CATACTCATAATAATTGTAATACTGAAACTGGTCAATGTATTTGTCCA
CCACATACTGCTGGTGTTAAATGTGAATTGTGTGAAGCTAATCATTGG
GGTCATGATTCTAAATTGGGTTGTAAACCATGTGATTGTTCTTTGGTT
GGTTCTTCTTCTTCTCAATGTGATTTGTCTTCTGGTTGGTGTCAATGT
AATTCTGAATTTGGTGCTGAAAAATGTAATGAATGTGCTTTGGGTTTT
AGAGCTTATCCAGAATGTACTGCTTGTAATTGTTCTATTATTGGTACT
AGAGAAGAATATTGTGATGAAAAACAAGGTGTTTGTGGTTGTGATAAA
GATTCTTCTACTTGTTCTTGTAAAGTTAATGTTGTTGGTCCAGGTTGT
GATGAATGTAAATTGGGTACTTTTGCTTTGTCTGCTGATGATCCATTG
GGTTGTTCTCCATGTTTTTGTTTTGGTGTTTCTGAAACTTGTGAAGAA
TTGGGTGGTTTGGTTAGAATTCCAATTATTTTGACTCCAGATCAAGAA
ACTTTGCATGTTGTTTCTCAATCTGATTTGTCTGGTACTTTGGATGGT
GTTTTTTTGCAATCTCCAGATATTTTGTTGGATGCTGCTTTGGTTAAA
CAATCTTTGCATACTGAACCATTTTATTGGAGATTGCCAGAACAATTT
CAAGGTAATAAATTGTTGGCTTATGGTGGTAAATTGAGATATACTGCT
GCTTTTTATGCTTTGGAAGGTTCTGGTATTTCTAATTTTGAACCACAA
TTGTTGATTAAAGGTGGTAGAACTTCTAAATTGAATATTTATAGAGAT
GTTCCAGCTCCAGATAATGGTGTTGAAACTTCTCAAGAAATTGATTTG
AAAGAAAGAGAATGGAAATATTTTAATTCTGTTTCTGATCAAGCTGTT
TCTCATTCTGATTTTATGTCTGTTTTGTCTAATATTCAATATATTTTG
ATTAAAGCTTCTTATGGTTCTGATTTGCAACAATCTAGAATTTCTAAT
ATTTCTTTGGAAATTGCTGTTGATTCTGATGAAATGAATGCTGGTAGA
GAAACTGCTAGAGGTATTGAAAGATGTAAATGTCCATCTGGTTATGCT
GGTTTGTCTTGTCAAGAATGTGCTCCAGGTTTTTATAGACAATCTTTG
ACTGAATTGAATGTTAGAGGTCCAAGACCATTGATTGAACCATCTTGT
AATGATAATTGTACTGGTGTTTTGTTGAATGATTTGGAAAATTTGGAA
TTGTCTATGCAATCTGTTAATTTGACTGGTGTTATTTTGGCTCCATAT
TCTTTGTTGGCTTCTTTGGAAAATACTACTGATGAAATTAAAATGTTG
TTGTCTCCAGAAAGAAATTCTTCTTTTTTGTTGAAAAAAGCTGAAGAA
CAATTGTCTGGTTTGACTAAAGATATTGATCAATTGCATCAAAAAACT
ACTCAAGCTTATGGTGATGGTCAAGATTTGAATAAATCTACTGAAAGA
ATGGTTAATCAATCTCAAGAATTGTTGGATTCTATTACTAAAGTTCAA
ACTGCTATTCAAGCTTTGGTTGAATTGGGTTCTAATTTGAATAATACT
TTGGGTAATAATTTGGTTTTGACTAATTCTACTCAATTGTTGGATGAA
GTTTCTGCTGTTTTGGAAAAAATGAGAAATAAAGCTTTTACTCAACAA
CATCAAAATGCTACTCATGAATTTAAAGCTGCTGAAGCTTTGTTGTTG
CAAGTTCAAAGAGAATTTCAAAAACCACAAAAAGATGTTATGGAATTG
AAAGAAAGAATTGTTACTATTTTGTCTGAACATTCTTCTAAATTGCAA
GATGCTCAAAATTTGGTTAATGAATCTTTTACTAAAGCTAATGAAACT
AATCATATTTTGCATGCTATGTCTTCTAATTTGGCTGTTTTTTATGAT
AAAAAACAAAATGTTTCTTCTAATCATTTTTTGACTACTGAAATGATT
GAAGAAGCTCAAGCTACTATGACTGAAGGTTTGAATATTGCTACTGAT
GTTGTTAATGCTACTTCTCAATTGGAAGGTCATAAAGATGAATTGGCT
TTGTGGAATCCAAAATTGAGAAAACAAGTTGATAATTTGGTTATGCAA
ATGACTAAAAGATCTGTTTTGGATTTGGTTTATAAAGCTGAAGATCAT
GCTGCTGGTTTGAAAAAATTGGCTGATTCTTTGCATAATGTTTTGTCT
GGTGTTAGAAATGTTTCTTTTAATGCTACTAATGCTGCTCAAGCTAAT
TCTAATATTAAAATTAATATTGAAAAAGCTGAAAGATTGGCTGAACAA
GCTAATTCTACTGTTGCTGCTGCTTGGTCTTTGGCTTTGTTTGCTGAT
CATTCTTTGAAAGATGCTGGTGCTAAATCTTTGCAACAATCTTCTAGA
TTGTTGTCTGAAGCTGCTGATTTGAATAATAAAACTAATGGTTTGATT
TCTGATTTGAATGGTTTGAAAGAACAAGTTGATAAAATTAGATATAAT
GCTCAAAATATTTCTAAACAATTGTCTGAACCATTGCAATTGTTGTCT
TCTATTCCAAATAATGTTTCTGTTAAAGTTTTGGAAGCTAAAGAACAT
GCTATGGGTGCTAATGTTTCTGCTGTTGCTGCTTTGCAACATTTGGAA
GATTTTTCTCAAAAATTGGAAGAATCTTCTTCTGCTATGATTAGAGCT
AATGAAACTGTTAAAAGAACTAATGAATTGTTTTCTGATTCTGCTAAA
ACTGCTAATGCTGCTGAAAAAAAAGTTCAAGAAGTTGAAACTCAAACT
AATTTGTTGTTTGAAAGATTGAAACCATTGAAAATGTTGGAAGATACT
TTGTCTAGAAATTTGTCTGAAATTAAAGAATTGATTGATCAAGCTAGA
AAACAAGCTGCTTCTATTAAAGTTGCTGTTGCTGCTGAACAAGATTGT
GTTAGAGCTTATAAACCAGATATTTCTTCTTCTAATTATAATACTTTG
ACTTTGATTGTTAAAACTTCTGAATCTGATAATTTGTTGTTTTATTTG
GGTTCTTCTACTAATGTTGATTTTATGGCTTTGGAAATGAGAAGAGGT
AAAGTTTCTTTTTTGTGGGATGTTGGTTCTGGTTTTGCTAAATTGGAA
TATCCAGATATTCAAATTAATAATGATAAATGGCATAGAATTCATGCT
ACTAGATTTGGTAAAACTGGTTCTTTGACTGTTCAAGAATTGAAATCT
TCTCAAACTCCAACTGTTAAAACTGCTACTTCTCCAGGTACTTCTACT
GTTTTGGATGTTAATAAATCTACTTTGGTTTTTGTTGGTGGTTTGGGT
GGTCAAATTAAAAAATCTTCTGCTGTTAAAATGACTCATTTTAAAGGT
TGTATGGGTGGTGCTTCTTTGAATGGTAATAATATTGGTTTGTGGAAT
TATGCTGAAAGAGAAGGTAAATGTAGAGGTTGTTTTATGTCTCCACAA
GATGAAGATACTTCTTTTCATTTTGATGGTTCTGGTTATTCTGTTGTT
GAAAAAGCTTTGAGATCTACTGTTACTCAAATTGTTATGTTGTTTAAA
ACTTTTTCTCCAAATGGTTTGTTGTTGTATTTGGCTTCTAATGGTACT
AGAGATTTTACTTCTATTGAATTGGTTGATGGTAAAGTTAGATTGACT
TTTGAATTGGGTTCTGGTCCATTGTCTTTGATTACTGATAAAACTTAT
AATTCTGGTAATTGGTATAAAATTGCTTTTCATAGAACTAAACAAAAA
GGTTATTTGGCTGTTATGGATGCTTATATTCCAACTAATAGAGAAACT
AAAGAAGGTACTACTTCTGGTTCTGCTTCTGATTTGAATAGATCTGAT
AAAGATCCAATTTATATTGGTGGTTTGCCAAGATCTAGACCAGTTAGA
AGACAATTGGTTACTAGATCTTATGTTGGTTGTATTAAAAATTTGGAA
ATTGCTAGATCTAATTTTGATTTGTTGAAAGAATCTTATGGTGTTAAA
AAAGGTTGTGTTTTGGAACATGGTACTGAACCATGGTAT.

### Stem Cell Factor (SCF) Polypeptide

In some embodiments, a composition of the invention comprises a stem cell factor (SCF) polypeptide. SCF is sometimes referred to in the literature as “Kit ligand”. Cosmetic benefits of SCF activity are believed to include recovering skin health, skin regeneration, and delaying aging of skin cells.

The corresponding GenBank reference (RXN01125.1) annotates SCF as being present in the blood.

A SCF polypeptide of the invention may be recombinant or synthetic. In some embodiments, a SCF polypeptide of the invention comprises an amino acid sequence having at least 70% sequence identity to SEQ ID NO: 8. In some embodiments, the SCF polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 8. In some embodiments, the SCF polypeptide of the invention has at least 75%, at least 80%, at least 85%, at least 90%, at least 91%, at least 92%, at least 93%, at least 94%, at least 95%, at least 96%, at least 97%, at least 98%, or at least 99% sequence identity to a functional fragment or catalytic domain of SEQ ID NO: 8.

In some embodiments, a SCF polypeptide of the invention comprises at least 50 consecutive amino acids of SEQ ID NO: 8. In some embodiments, a SCF polypeptide of the invention comprises at least 50, at least 60, at least 70, at least 80, at least 90, at least 100, at least 110, at least 120, at least 130, at least 140, at least 150, at least 160, at least 170, at least 180, at least 190, at least 200, at least 225, at least 250, at least 275, at least 300, at least 310, at least 311, at least 312, at least 313, or at least 314 consecutive amino acids of SEQ ID NO: 8. In some embodiments, a SCF polypeptide of the invention comprises at least 50, at least 60, at least 70, at least 80, at least 90, at least 100, at least 110, at least 120, at least 130, at least 140, at least 150, at least 160, at least 170, at least 180, at least 190, at least 200, at least 225, at least 250, at least 275, at least 300, at least 310, at least 311, at least 312, at least 313, or at least 314 consecutive amino acids of SEQ ID NO: 8, wherein the consecutive amino acids comprise the catalytic domain of the a SCF polypeptide.

The SCF polypeptide sequence is represented by SEQ ID NO: 8:

(SEQ ID NO: 8)
MYLFPQIWITAFIYPCLLFCFTFVEHSCGLGNVVTDDVNKIPILKGNI
PNDYIIKVRYVPQSEELNDICWLMLNIYELQLSLTSLAVKFAETSSNK
ENITVLIDMVMKMRRPFQYEEEVIDDYHCHYQDGNFNTSVYFDYLKKM
IETYELHERQFISPDCVSPPCPTSETTTLVAGSITIATELLIMNTTAC
VSASDCNTQETRRDKNTEAKTNTQGAEKLHIPLYLLLIPVFGILLVLT
WKGHITSCCRTIVLKGVDVASNTWVTLYMLLPDEAVLYKSTYSSFIWS
EGQKLDSCSVELLSETRVCKTSTPEIQ.

A SCF polypeptide of the invention may have one or more (e.g., up to 2, 3, 5, 10, 20, 30, 40, or 50) conservative amino acid substitutions relative to the polypeptide of SEQ ID NO: 8.

The invention also provides a fusion protein that includes at least a portion (e.g., a fragment or domain) of a SCF polypeptide of the invention attached to one or more fusion segments, which are typically heterologous to the SCF polypeptide.

A SCF nucleic acid of the invention encodes a SCF polypeptide of the invention. The SCF nucleic acid is typically an isolated nucleic acid. A SCF nucleic acid of the invention may be optimized for expression in a recombinant cell.

In some embodiments, the SCF nucleic acid sequence has at least 70% sequence identity to SEQ ID NO: 12. In some embodiments, the SCF nucleic acid sequence has at least 75%, at least 80%, at least 85%, at least 90%, at least 95%, at least 96%, at least 97%, at least 98%, or at least 99%, or 100% sequence identity to SEQ ID NO: 12, over a region of at least about 10, e.g., at least about 15, 20, 25, 30, 35, 40, 45, 50, 75, 100, 150, 200, 250, 300, 350, 400, 450, 500, 550, 600, 650, 700, 750, 800, 850, 900, or 945 nucleotides, or a catalytic domain thereof.

SEQ ID NO: 12 represents a nucleic acid sequence corresponding to the SCF polypeptide (SEQ ID NO: 8) with a S. cerevisiae codon bias, to optimize for expression in Pichia pastoris:

(SEQ ID NO: 12)
ATGTATTTGTTTCCACAAATTTGGATTACTGCTTTTATTTATCCATGT
TTGTTGTTTTGTTTTACTTTTGTTGAACATTCTTGTGGTTTGGGTAAT
GTTGTTACTGATGATGTTAATAAAATTCCAATTTTGAAAGGTAATATT
CCAAATGATTATATTATTAAAGTTAGATATGTTCCACAATCTGAAGAA
TTGAATGATATTTGTTGGTTGATGTTGAATATTTATGAATTGCAATTG
TCTTTGACTTCTTTGGCTGTTAAATTTGCTGAAACTTCTTCTAATAAA
GAAAATATTACTGTTTTGATTGATATGGTTATGAAAATGAGAAGACCA
TTTCAATATGAAGAAGAAGTTATTGATGATTATCATTGTCATTATCAA
GATGGTAATTTTAATACTTCTGTTTATTTTGATTATTTGAAAAAAATG
ATTGAAACTTATGAATTGCATGAAAGACAATTTATTTCTCCAGATTGT
GTTTCTCCACCATGTCCAACTTCTGAAACTACTACTTTGGTTGCTGGT
TCTATTACTATTGCTACTGAATTGTTGATTATGAATACTACTGCTTGT
GTTTCTGCTTCTGATTGTAATACTCAAGAAACTAGAAGAGATAAAAAT
ACTGAAGCTAAAACTAATACTCAAGGTGCTGAAAAATTGCATATTCCA
TTGTATTTGTTGTTGATTCCAGTTTTTGGTATTTTGTTGGTTTTGACT
TGGAAAGGTCATATTACTTCTTGTTGTAGAACTATTGTTTTGAAAGGT
GTTGATGTTGCTTCTAATACTTGGGTTACTTTGTATATGTTGTTGCCA
GATGAAGCTGTTTTGTATAAATCTACTTATTCTTCTTTTATTTGGTCT
GAAGGTCAAAAATTGGATTCTTGTTCTGTTGAATTGTTGTCTGAAACT
AGAGTTTGTAAAACTTCTACTCCAGAAATTCAA.

An example of an algorithm that is suitable for determining sequence similarity is the BLAST algorithm, which is described in Altschul et al., 1990, J. Mol. Biol. 215:403-410. Software for performing BLAST analyses is publicly available through the National Center for Biotechnology Information. This algorithm involves first identifying high scoring sequence pairs (HSPs) by identifying short words of length W in the query sequence that either match or satisfy some positive-valued threshold score T when aligned with a word of the same length in a database sequence. These initial neighborhood word hits act as starting points to find longer HSPs containing them. The word hits are expanded in both directions along each of the two sequences being compared for as far as the cumulative alignment score can be increased. Extension of the word hits is stopped when: the cumulative alignment score falls off by the quantity X from a maximum achieved value; the cumulative score goes to zero or below; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff & Henikoff, 1992, Proc. Nat'l. Acad. Sci. USA 89:10915-10919) alignments (B) of 50, expectation (E) of 10, M′5, N′-4, and a comparison of both strands.

Any of the amino acid sequences described herein can be produced together or in conjunction with at least 1, e.g., at least (or up to) 2, 3, 5, 10, or 20 heterologous amino acids flanking each of the C- and/or N-terminal ends of the specified amino acid sequence, and or deletions of at least 1, e.g., at least (or up to) 2, 3, 5, 10, or 20 amino acids from the C- and/or N-terminal ends.

Conservative substitutions can be chosen from among a group of amino acids having a similar side chain to the reference amino acid. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Accordingly, exemplary conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

Suitable fusion segments include, without limitation, segments that can provide other desirable biological activity or facilitate purification of the polypeptide(s) of the invention (e.g., by affinity chromatography). Fusion segments can be joined to the amino or carboxy terminus of polypeptide(s) of the invention. The fusion segments can be susceptible to cleavage. In some embodiments, the fusion segment is a tag e.g. a purification tag. In some embodiments, the purification tag is a His-tag (e.g. a 6× His-tag or a 8× His-tag).

“Optimized” nucleic acid sequences encode an amino acid sequence using codons that are preferred in a recombinant cell. The optimized nucleic acid sequence is typically engineered to retain completely or as much as possible of the amino acid sequence originally encoded by the starting nucleic acid sequence, which is also known as the “parental” sequence.

Several methods for codon optimization are known in the art. Preferably, codon optimized sequences avoid nucleotide repeats and restriction sites that are utilized in cloning the nucleic acids of the invention, by adjusting the settings in commercial software or by manually altering the sequences to substitute codons that introduce undesired sequences, for example with highly utilized codons in the heterologous organism of interest.

In some embodiments, the nucleic acid is codon optimized for expression in a prokaryotic cell. In some embodiments, the nucleic acid is codon optimized for expression in Acinetobacter, Agrobacterium, Escherichia, Cupriavidus, Clostridium, Rhodobacter, Marinobacter, Bacillus, Klebsiella, Tatumella, Pseudomonas, Ralstonia, Rhodococcus, Methylobacterium, Methylophilus, Methylococcus, Methylomicrobium, Methylomonas, Pantoea, Streptomyces, Parachlorella, Synechococcus, Synechocystis and Thermocynechococcus. In some embodiments, the nucleic acid is codon optimized for expression in E. coli.

In some embodiments, the nucleic acid is codon optimized for expression in a eukaryotic cell. In some embodiments, the nucleic acid is codon optimized for expression in a yeast cell, a fungal cell, an algal cell and a plant cell. In some embodiments, the nucleic acid is codon optimized for expression in Pichia, Saccharomyces, Kluyveromyces, Candida, Schizosaccharomyces, Schefferomyces, Rhodosporidium, Hansenula, Klockera, Schwanniomyces, Issatchenkia, Yarrowia or Rhodotorula. In some embodiments, the nucleic acid is codon optimized for expression in S. cerevisiae, C. lipolytica, R. glutinis, S. bulderi, S. barnetti, S. exiguus, S. uvarum, S. diastaticus, K. lactis, K. marxianus K. fragile, P. kudriavzevii, S. stipitis or I. orientalis.

In some embodiments (e.g. where codon optimization is not well-established for a particular cell type), the nucleic acid is codon optimized for expression in a similar cell type. For example, in some embodiments, the nucleic acid sequence is optimized with a S. cerevisiae codon bias, to optimize for expression in Pichia pastoris.

In some embodiments, the nucleic acid is codon optimized for expression in a filamentous fungal cell. In some embodiments, the nucleic acid is codon optimized for expression in Aspergillus, Penicillium, Rhizopus, Chrysosporium, Myceliophthora, Trichoderma, Humicola, Acremonium or Fusarium. In some embodiments, the nucleic acid is codon optimized for expression in A. niger, A. oryzae, T. reesei, P. chrysogenum, M. thermophila, or R. oryzae.

In some embodiments, the nucleic acid is codon optimized for expression in Botryococcus, Nannochloropsis, Chlorella, Chlamydomonas, Dunaliella, Chaetoceros, Porphyridium, Scenedesmus or Pseudochlorococcum. In some embodiments, the nucleic acid is codon optimized for expression in B. braunii or N. gaditana.

### Vectors

A vector of the invention comprises a nucleic acid of the invention. A vector may comprise one or more of an origin of replication, a promoter sequence operably linked to a nucleic acid of the invention and a reporter gene or selectable marker. Any suitable vector may be used e.g. a pPIC9K vector.

The promoter may be homologous or heterologous. The promoter may be constitutive or inducible.

In one embodiment, the promoter is inducible and is activated in the presence of an inducing agent. Inducing agents include, but are not limited to, sugars, metal salts, and antibiotics. In some embodiments, the inducible promoter is an AOX1 promoter. In some cases, the promoter allows constitutive expression of the polypeptide(s) of the invention.

In one embodiment, promoters that are active at different stages of growth can be used.

The promoter sequence may be operable in a prokaryotic cell, for example an E. coli cell.

The promoter sequence may be operable in a eukaryotic cell, for example a yeast cell, a fungal cell, an algal cell or a plant cell.

Where the recombinant cell is a fungal cell, the promoter can be a fungal promoter (including, but not limited to, a filamentous fungal promoter), a promoter operable in plant cells, or a promoter operable in mammalian cells. Mammalian, mammalian viral, plant and plant viral promoters can drive particularly high expression when the associated 5′ UTR sequence (i.e., the sequence which begins at the transcription start site and ends one nucleotide before the start codon), normally associated with the mammalian or mammalian viral promoter is replaced by a fungal 5′ UTR sequence. The source of the 5′ UTR can vary provided it is operable in the filamentous fungal cell. In various embodiments, the 5′ UTR can be derived from a yeast gene or a filamentous fungal gene. The 5′ UTR can be from the same species as the recombinant cell or from a different species.

Promoters for recombinant expression in yeast are known in the art. Suitable promoters for S. cerevisiae include, but are not limited to, the MFα1 promoter, galactose inducible promoters such as GAL1, GAL7, and GAL10 promoters, glycolytic enzyme promoters including the TPI and PGK promoters, the TDH3 promoter, the TEF1 promoter, the TRP1 promoter, the CYCI promoter, the CUP1 promoter, the PHO5 promoter, the ADH1 promoter, and the HDP promoter. A suitable promoter in the genus Pichia sp. is the AOXI promoter.

Suitable reporter genes or selectable markers include, but are not limited to, a drug resistance gene, a metabolic enzyme, a factor required for survival of the recombinant cell, a fluorescent marker, or an enzyme that generates a detectable product. Cells transformed with the vector can be selected based on their ability to grow in the presence of inhibitors (e.g. antibiotics) or under conditions in which untransformed cells cannot grow.

The vector may be a high copy number vector, an intermediate copy number vector, or a low copy number vector.

### Recombinant Cells

The invention provides recombinant cells engineered to express a polypeptide of the invention. The invention also provides recombinant cells transformed with a nucleic acid of the invention. The nucleic acid may be extrachromosomal, on a vector (typically a plasmid). In some embodiments, the recombinant cell is transformed with a vector of the invention. In some embodiments, the recombinant cell is transiently transformed. Alternatively, the recombinant cell may be stably transformed wherein the nucleic acid is integrated in one or more copies into the genome of the cell. Integration into the cell's genome may occur at random by non-homologous recombination but preferably, the nucleic acid construct may be integrated into the cell's genome by homologous recombination, as is well known in the art.

In some embodiments, the recombinant cell is a prokaryotic cell. In some embodiments, the prokaryotic cell is selected from Acinetobacter, Agrobacterium, Escherichia, Cupriavidus, Clostridium, Rhodobacter, Marinobacter, Bacillus, Klebsiella, Tatumella, Pseudomonas, Ralstonia, Rhodococcus, Methylobacterium, Methylophilus, Methylococcus, Methylomicrobium, Methylomonas, Pantoea, Streptomyces, Parachlorella, Synechococcus, Synechocystis and Thermocynechococcus. In some embodiments, the prokaryotic cell is E. coli. Suitable cells of the bacterial genera include, but are not limited to, cells of Lactobacillus, Pseudomonas, and Streptomyces. Suitable cells of bacterial species include, but are not limited to, cells of Bacillus subtilis, Bacillus licheniformis, Lactobacillus brevis, Pseudomonas aeruginosa, and Streptomyces lividans.

In some embodiments, the recombinant cell is a eukaryotic cell. In some embodiments, the eukaryotic cell is selected from the group consisting of a yeast cell, a fungal cell, an algal cell and a plant cell. In some embodiments, the yeast cell is selected from the group consisting of Pichia, Saccharomyces, Kluyveromyces, Candida, Schizosaccharomyces, Schefferomyces, Rhodosporidium, Hansenula, Klockera, Schwanniomyces, Issatchenkia, Yarrowia and Rhodotorula. In some embodiments, the yeast cell is selected from the group consisting of P. pastoris, S. cerevisiae, C. lipolytica, R. glutinis, S. bulderi, S. barnetti, S. exiguus, S. uvarum, S. diastaticus, K. lactis, K. marxianus, K. fragile, P. kudriavzevii, S. stipitis and I. orientalis. Suitable cells of yeast include, but are not limited to C. albicans, S. pombe, H. polymorpha, P. canadensis, or P. rhodozyma.

In some embodiments, the fungal cell is a filamentous fungal cell. In some embodiments, the filamentous fungal cell is selected from the group consisting of Aspergillus, Penicillium, Rhizopus, Chrysosporium, Myceliophthora, Trichoderma, Humicola, Acremonium and Fusarium. In some embodiments, the filamentous fungal cell is selected from the group consisting of A. niger, A. oryzae, T. reesei, P. chrysogenum, M. thermophila, and R. oryzae. Suitable cells of filamentous fungal genera include, but are not limited to, cells of Aureobasidium, Bjerkandera, Ceriporiopsis, Coprinus, Coriolus, Corynascus, Chaetomium, Cryptococcus, Filobasidium, Gibberella, Hypocrea, Magnaporthe, Mucor, Neocallimastix, Neurospora, Paecilomyces, Phanerochaete, Phlebia, Piromyces, Pleurotus, Scytaldium, Schizophyllum, Sporotrichum, Talaromyces, Thermoascus, Thielavia, Tolypocladium and Trametes. In certain aspects, the recombinant cell is a Trichoderma sp., Penicillium sp., Humicola sp. (e.g., Humicola insolens); Aspergillus sp., Chrysosporium sp., Fusarium sp., or Hypocrea sp.. Suitable cells can also include cells of various anamorph and teleomorph forms of these filamentous fungal genera.

Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum, Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, and Trichoderma viride.

In some embodiments, the algal cell is selected from the group consisting of Botryococcus, Nannochloropsis, Chlorella, Chlamydomonas, Dunaliella, Chaetoceros, Porphyridium, Scenedesmus and Pseudochlorococcum. In some embodiments, the algal cell is selected from the group consisting of B. braunii and N. gaditana.

Recombinant cells may be cultured in conventional nutrient media modified as appropriate for activating promoters (if an inducible promoter is present), selecting transformants, or amplifying the nucleic acid sequence encoding the polypeptide(s) of the invention. Culture conditions, such as temperature, pH and the like, are those previously used with the recombinant cell selected for expression, and will be apparent to those skilled in the art. Preferred culture conditions for a given recombinant cell may be found in the scientific literature and/or from the source of the recombinant cell such as the American Type Culture Collection (ATCC).

### BRIEF DESCRIPTION OF THE FIGURES

FIG. 1. pPIC9K vector map.

FIG. 2. Process for purifying proteins by His-affinity chromatography.

### EXAMPLES

The invention will now be described with reference to the following non-limiting examples.

In an attempt to identify the polypeptide repertoire within caviar, the inventors subjected caviar (roe) and roe sacks, as well as caviar oil to analysis by liquid chromatography-mass spectrometry (LC-MS). Analysed samples were from Acipenser baerii.

Significant technical challenges were encountered throughout the LC-MS analysis, which were overcome by the development of a bespoke sample preparation technique. The ammonium sulphate precipitate method involved making a biphasic solution of the oil and water (equal volumes), to which was added solid ammonium sulphate, such that it was 90% (w/v) saturation (˜0.6 g/ml). This was agitated for 1 hour in a cold room. The solution was then centrifuged at 20000×g for 30 min at 4° C., the oil top layer was removed, the lower aqueous layer was removed carefully, leaving behind the precipitated proteins. The pellet was suspended in a minimal volume of lysis buffer and stored on ice or at 4° C. for later analysis by SDS-PAGE. Urea isolation of proteins involved preparation of a 5M urea solution, which was added to oil to yield a ratio of 1:3 (aqueous:oil). This emulsion was agitated over night at 4° C. and then centrifuged at 20000×g for 30 min at 4° C. The oil top layer was removed, the lower aqueous layer (5M Urea) was removed, containing precipitate and denatured proteins. A minimal volume of lysis buffer was added and stored on ice or at 4° C. for later analysis by SDS-PAGE. The samples were run in a protein-gel. Once into the resolving gel, the gel was stopped, stained and the single band containing all the protein material was subjected to LC-MS and mass fingerprinting. Without extensive published genomic data for Acipenser baerii, the results of this analysis identified proteins from A. ruthenus as a close sequence match.

Having identified the polypeptide repertoire within caviar, the inventors analysed the cellular functions associated with each of the hits, and determined which of those proteins possess function(s) which are desirable in cosmetic applications. Those key caviar polypeptides include: SOD3, SOD1, TIMP1, aFGF, bFGF, IGF-2 and laminin (details of which are provided above).

SOD1 (SEQ ID NO: 2), SOD3 (SEQ ID NO: 1) and SCF (SEQ ID NO: 8) were expressed in P. pastoris. Briefly, codon optimised genes were subcloned into pPIC9K vectors via EcoR I and Not I enzyme digestion sites. Genes were expressed under the control of a AOX1 promoter. Expressed proteins included a C-terminal 8× His-tag to aid purification.

The SOD3 polypeptide of SEQ ID NO: 1 comprising a C-terminal 8× His-tag is represented by SEQ ID NO: 19:

(SEQ ID NO: 19)
MTMSAFSFLLALAIAGTHVSHSEESPTSEENTMKNIESKVNDLWQSLL
HPVAFVAKDAELVYASCEMKPSTKLEEGKPQVTGKVLFKQAYPQGRLE
SIINLEGFPKTSNQSRAIHIHEFGDLSDGCDAAGGHFNPFKVNHPRHP
GDFGNFLPKNSQIKTLKKNIQATMFGPNSFLSRSVVIHELKDDLGKGD
NPASLLNGNAGKRLACCVIGISNKNLWEKTSQSLTSSKKKRNARGLAN
KQAHHHHHHHH.

The SOD1 polypeptide of SEQ ID NO: 2 comprising a C-terminal 8× His-tag is represented by SEQ ID NO: 20:

(SEQ ID NO: 20)
MVLKAVCVLKGTGDVCGTVHFVQEKEAGPVKLTGQITGLTPGEHGFHV
HAFGDNTNGCASAGPHFNPLGKTHGAPQDEIRHIGDLGNVIAGDDKVA
IINIEDKLITLSGAYSIIGRTMVIHEKADDLGKGGNDESLVTGNAGGR
LACGVIGIAQSHHHHHHHH.

The SCF polypeptide of SEQ ID NO: 8 comprising a C-terminal 8× His-tag is represented by SEQ ID NO: 21:

(SEQ ID NO: 21)
MYLFPQIWITAFIYPCLLFCFTFVEHSCGLGNVVTDDVNKIPILKGNI
PNDYIIKVRYVPQSEELNDICWLMLNIYELQLSLTSLAVKFAETSSNK
ENITVLIDMVMKMRRPFQYEEEVIDDYHCHYQDGNFNTSVYFDYLKKM
IETYELHERQFISPDCVSPPCPTSETTTLVAGSITIATELLIMNTTAC
VSASDCNTQETRRDKNTEAKTNTQGAEKLHIPLYLLLIPVFGILLVLT
WKGHITSCCRTIVLKGVDVASNTWVTLYMLLPDEAVLYKSTYSSFIWS
EGQKLDSCSVELLSETRVCKTSTPEIQHHHHHHHH.

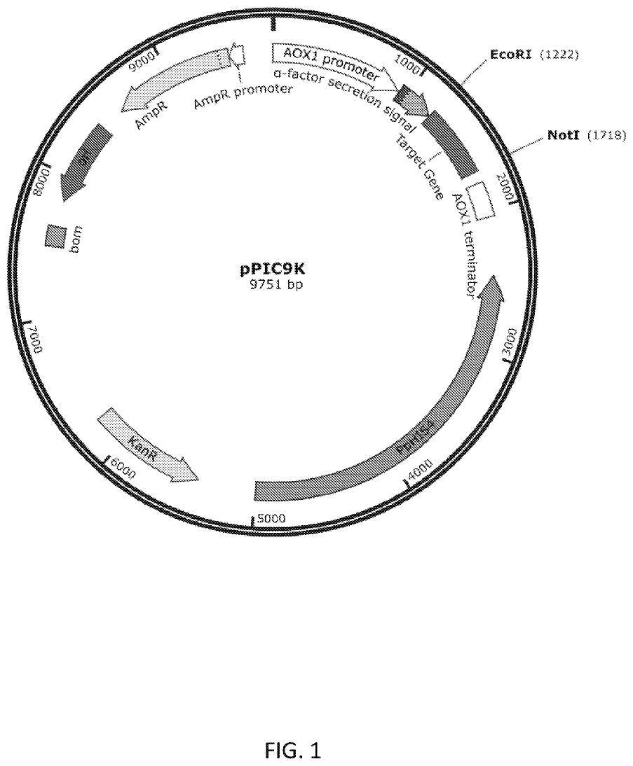

A pPIC9K vector map is provided in FIG. 1.

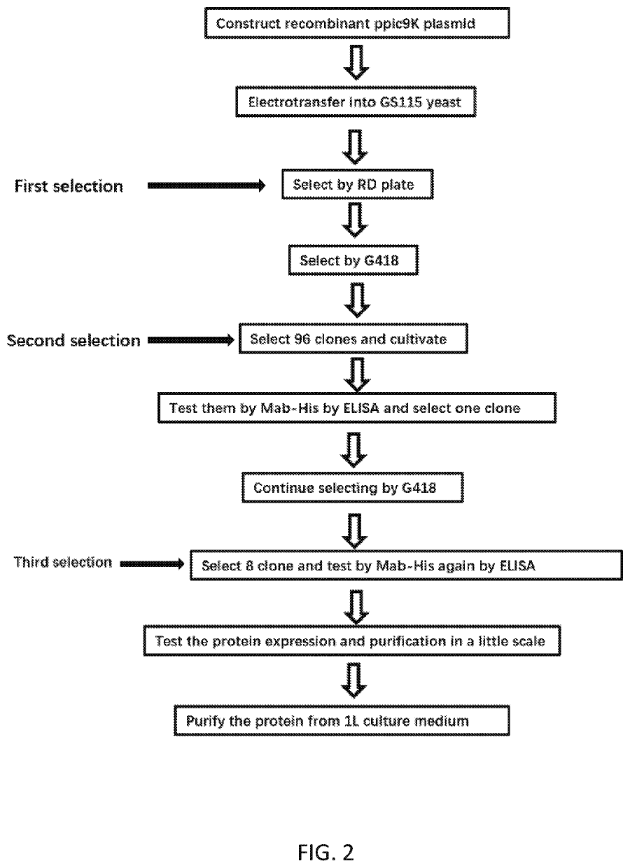

Positive clones were identified based on G418 resistance; followed by small-scale expression in P. pastoris. Proteins were purified via His-affinity chromatography, according to the process summarised in FIG. 2.

To prepare the exemplified cosmetic compositions, a stock solution of protein mix (comprising SOD1, SOD3 and SCF) was prepared in 5 mM sodium phosphate buffer at pH 6.0. The concentration of each protein was 0.04 mg/ml. The stock solution was diluted in 5 mM sodium phosphate at pH 6.0 to a final concentration (of each protein) of 400 microgr/l.

1. A composition comprising a recombinant or synthetic caviar polypeptide, wherein the caviar polypeptide is selected from:
a) a superoxide dismutase (SOD3) polypeptide, wherein the SOD3 polypeptide comprises a polypeptide sequence having: (i) at least 70% sequence identity to the amino acid sequence of SEQ ID NO: 1; or (ii) at least 50 consecutive amino acids of SEQ ID NO: 1;
b) a superoxide dismutase (SOD1) polypeptide, wherein the SOD1 polypeptide comprises a polypeptide sequence having: (i) at least 70% sequence identity to the amino acid sequence of SEQ ID NO: 2; or (ii) at least 50 consecutive amino acids of SEQ ID NO: 2;
c) a tissue inhibitor of metalloproteinase 1 (TIMP1) polypeptide, wherein the TIMP1 polypeptide comprises a polypeptide sequence having: (i) at least 70% sequence identity to the amino acid sequence of SEQ ID NO: 3; or (ii) at least 50 consecutive amino acids of SEQ ID NO: 3;
d) an acidic fibroblast growth factor (aFGF) polypeptide, wherein the aFGF polypeptide comprises a polypeptide sequence having: (i) at least 70% sequence identity to the amino acid sequence of SEQ ID NO: 4; or (ii) at least 50 consecutive amino acids of SEQ ID NO: 4;
e) a basic fibroblast growth factor (bFGF) polypeptide, wherein the bFGF polypeptide comprises a polypeptide sequence having: (i) at least 70% sequence identity to the amino acid sequence of SEQ ID NO: 5; or (ii) at least 50 consecutive amino acids of SEQ ID NO: 5;
f) an insulin-like growth factor 2 (IGF-2) polypeptide, wherein the IGF-2 polypeptide comprises a polypeptide sequence having: (i) at least 70% sequence identity to the amino acid sequence of SEQ ID NO: 6; or (ii) at least 50 consecutive amino acids of SEQ ID NO: 6; and
g) a laminin polypeptide, wherein the laminin polypeptide comprises a polypeptide sequence having: (i) at least 70% sequence identity to the amino acid sequence of SEQ ID NO: 7; or (ii) at least 50 consecutive amino acids of SEQ ID NO: 7.

2. The composition according to claim 1, wherein the composition comprises two or more recombinant or synthetic caviar polypeptides.

3. The composition according to claim 1 or claim 2, wherein the composition comprises two or more recombinant or synthetic caviar polypeptides selected from: SOD3, SOD1 and TIMP1.

4. The composition according to any one of the preceding claims, wherein the composition comprises a recombinant or synthetic SOD3 polypeptide and a recombinant or synthetic SOD1 polypeptide.

5. The composition according to any one of the preceding claims, wherein the composition further comprises a recombinant or synthetic stem cell factor (SCF) polypeptide, wherein the SCF polypeptide comprises a polypeptide sequence having: (i) at least 70% sequence identity to the amino acid sequence of SEQ ID NO: 8; or (ii) at least 50 consecutive amino acids of SEQ ID NO: 8.

6. The composition according to any one of the preceding claims, wherein the composition comprises a recombinant or synthetic SOD3 polypeptide, a recombinant or synthetic SOD1 polypeptide, and a recombinant or synthetic SCF polypeptide.

7. The composition according to any one of the preceding claims, wherein one or more of the recombinant or synthetic polypeptides comprise a purification tag, such as a His-tag.

8. The composition according to any one of the preceding claims, wherein the composition further comprises collagen.

9. The composition according to any one of the preceding claims, wherein the composition is a cosmetic composition.

10. The composition according to claim 9, wherein the cosmetic composition is selected from: skin moisturizer, perfume, lipstick, fingernail polishes, eye and/or facial makeup preparation, shampoo, hair colouring preparations, toothpaste, and deodorant.

11. An isolated nucleic acid which encodes one or more caviar polypeptide(s) as defined in claim 1.

12. An isolated nucleic acid which encodes SCF as defined in claim 5.

13. The isolated nucleic acid according to claim 11 or claim 12, wherein the nucleic acid is codon optimized for expression in a eukaryotic cell.

14. The isolated nucleic acid according to claim 13, wherein the eukaryotic cell is a yeast cell.

15. The isolated nucleic acid according to claim 14, wherein:
a) the nucleic acid encoding SOD3 has at least 70% sequence identity to SEQ ID NO: 9;
b) the nucleic acid encoding SOD1 has at least 70% sequence identity to SEQ ID NO: 10;
c) the nucleic acid encoding TIMP1 has at least 70% sequence identity to SEQ ID NO: 11; and
d) the nucleic acid encoding SCF has at least 70% sequence identity to SEQ ID NO: 12.

16. A vector comprising the isolated nucleic acid according to any one of claims 11-15.

17. The vector according to claim 16, further comprising:
(a) an origin of replication;
(b) a promoter sequence operably linked to said nucleic acid; and/or
(c) a reporter gene.

18. A recombinant cell engineered to express one or more recombinant or synthetic caviar polypeptide(s) according to any one of claim 1-4, 6 or 7.

19. A recombinant cell transformed with the vector according to claim 16 or claim 17.

20. The recombinant cell according to claim 18 or claim 19, wherein the recombinant cell is a P. pastoris cell.

21. A method of producing one or more recombinant caviar polypeptides according to any one of claim 1-4, 6 or 7, the method comprising:
(a) culturing a recombinant cell according to any one of claims 18-20 in a suitable culture medium; and
(b) allowing expression of the recombinant caviar polypeptide.

22. A method of producing one or more synthetic caviar polypeptides according to any one of claim 1-4, 6 or 7, wherein the synthetic caviar polypeptides are produced by:
(a) cell-free protein synthesis; or
(b) solid-phase chemical synthesis.

23. A method of producing a cosmetic product, the method comprising combining:
(a) one or more recombinant or synthetic caviar polypeptides according to any one of claim 1-4, 6 or 7, optionally in combination with recombinant or synthetic SCF according to claim 5 or 7; with
(b) ingredients for a cosmetic product.

24. Use of the composition according to any one of claims 1-10 for improving the appearance of skin and/or hair.

25. A method of improving the appearance of skin, the method comprising administering to the skin of a subject a composition according to any one of claims 1-10.

26. A method of improving the appearance of hair or a subject, the method comprising administering to the hair of a subject a composition according to any one of claims 1-10.

27. A cosmetic method comprising administering the composition according to any one of claims 1-10 to the skin and/or hair of a subject.
